(19) United States
(12) Patent Application Publication
(10) Pub. No.: US 2009/0010909 A1
(43) Pub. Date: Jan. 8, 2009

(54) Novel GPCR and methods of use of the same

(75) Inventor: Pilar Ruiz-Lozano, San Diego, CA (US)

Correspondence Address: Biotechnology Law Group;c/o Portfolioip, P.O. Box 52050, Minneapolis, MN, 55402, US

(73) Assignee: BURNHAM INSTITUTE FOR MEDICAL RESEARCH, La Jolla, CA (US)

(21) Appl. No.: 12/151,617

(22) Filed: May 7, 2008

Related U.S. Application Data
(60) Provisional application No. 60/928,123, filed on May 7, 2007.

(51) Int. Cl. A61K 38/43; C12Q 1/02; C12Q 1/68; A61P 25/00; A01K 67/027; A61K 31/7088

(52) U.S. Cl. 424/94.1; 435/29; 435/6; 800/3; 514/44

(57) ABSTRACT

The present invention relates to methods of identifying antagonists of GPR22, a G protein-coupled receptor (GPCR) and related compositions and methods. Antagonists of GPR22 are useful as therapeutic agents for the treatment of ischemic heart disease, including myocardial infarction, post-myocardial infarction remodeling, and congestive heart failure.

### CROSS-REFERENCE TO RELATED CASES

This application claims priority from U.S. provisional patent application Ser. No. 60/928,123, filed 7 May 2007, which is incorporated herein by reference in its entirety.

### STATEMENT OF GOVERNMENT RIGHTS

This invention was made with Government support under 7 R01 HL065484-05 awarded by the National Institutes of Health. The Government has certain rights in the invention.

### TECHNICAL FIELD

The present invention relates to G protein coupled receptors (GPCRs) and their use.

### BACKGROUND INFORMATION

### Ischemic Heart Disease and Congestive Heart Failure

Congestive heart failure (CHF) affects nearly five million Americans with over 500,000 new cases diagnosed annually. By definition, CHF is a clinical syndrome in which heart disease reduces cardiac output, increases venous pressures, and is accompanied by molecular abnormalities that cause progressive deterioration of the failing heart and premature myocardial cell (myocyte) death (From; Heart Failure: Pathophysiology, Molecular Biology, and Clinical Management, Katz, A M, Lippincott Williams and Wilkins, 2000). In the adult heart, myocyte (cardiomyocyte) death is a critical element of the natural history of heart failure because the cells that are lost cannot be replaced. Current research from many groups has focused on the molecular mechanisms and signaling pathways that regulate myocyte death and survival.

Cell culture and small animal studies have clearly demonstrated that G-protein coupled receptors on cardiac myocytes are highly important regulators of cardiac contractile function and are also involved in the regulation of myocyte death and survival (for review, see Adams and Brown, Oncogene 20:1626-1634, 2001). However, there are no drugs currently available in the clinic designed to inhibit cardiac myocyte death or directly activate survival pathways. Recently published evidence in mice and rats demonstrate that activation of survival pathways (Lee et al., Endocrinol. 140:4831-40, 1999) or inhibitors of cardiac myocyte death pathways (Laugwitz et al., Human Gene Ther. (2001) 12:2051-2063, 2001) significantly improves cardiac function and animal survival.

Thus it is clear that similar therapeutic strategies for the treatment of human heart failure hold great promise.

### G Protein-Coupled Receptors

Although a number of receptor classes exist in humans, by far the most abundant and therapeutically relevant is represented by the G protein-coupled receptor (GPCR) class. It is estimated that there are some 30,000-40,000 genes within the human genome, and of these, approximately 2% are estimated to code for GPCRs. Receptors, including GPCRs, for which the endogenous ligand has been identified, are referred to as “known” receptors, while receptors for which the endogenous ligand has not been identified are referred to as “orphan” receptors.

GPCRs represent an important area for the development of pharmaceutical products: from approximately 20 of the 100 known GPCRs, approximately 60% of all prescription pharmaceuticals have been developed.

GPCRs share a common structural motif, having seven sequences of between 22 to 24 hydrophobic amino acids that form seven alpha helices, each of which spans the membrane (each span is identified by number, i.e., transmembrane-1 (IM-1), transmembrane-2 (TM-2), etc.). The transmembrane helices are joined by strands of amino acids between transmembrane-2 and transmembrane-3, transmembrane-4 and transmembrane-5, and transmembrane6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and membrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor), there is a change in the conformation of the receptor that facilitates coupling between the intracellular region and an intracellular “G-protein.”It has been reported that GPCRs are “promiscuous” with respect to G proteins, i.e., that a GPCR can interact with more than one G protein. See, Kenakin, T., 43 Life Sciences 1095 (1988). Although other G proteins may exist, currently, Gq, Gs, Gi, Gz and Go are G proteins that have been identified. Ligand-activated GPCR coupling with the G-protein initiates a signaling cascade process (referred to as “signal transduction”). Under normal conditions, signal transduction ultimately results in cellular activation or cellular inhibition. Although not wishing to be bound to theory, it is thought that the IC-3 loop as well as the carboxy terminus of the receptor interact with the G protein.

Gs-coupled GPCRs elevate intracellular cAMP levels. Gi-, Go-, or Gz-coupled GPCRs lower intracellular cAMP levels. Gq-coupled GPCRs elevate intracellular IP₃ and Ca²⁺ levels.

There are also promiscuous G proteins, which appear to couple several classes of GPCRs to the phospholipase C pathway, such as G15 or G16 (Offermanns and Simon, J. Biol. Chem. 270:15175-15180, 1995), or chimeric G proteins designed to couple a large number of different GPCRs to the same pathway, e.g. phospholipase C (Milligan and Rees, Trends in Pharm. Sci. 20:118-124, 1999).

Under physiological conditions, GPCRs exist in the cell membrane in equilibrium between two different conformations: an “inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

GPCR Expression in the heart. Cell culture and small animal studies have clearly demonstrated that G-protein coupled receptors on cardiac myocytes are highly important regulators of cardiac contractile function and are also involved in the regulation of myocyte hypertrophy (for review see; Adams and Brown, Oncogene, 20, 1626-1634, 2001). In fact, the positive effects of ACE inhibitors for treatment of CHF in humans is thought to at least partially involve the reduction of maladaptive hypertrophy via indirect inhibition of angiotensin II receptor activation in the myocardium. GPCR expression in cardiac myocytes and fibroblasts is reviewed in Tang and Insel, Trends Cardiovasc. Med. 14:94-99, 2004, which is incorporated herein by reference in its entirety. Only a fraction of the GPCRs identified in human genome databases have a known function; the majority are “orphan” receptors, that is, receptors without a known “parent” ligand. For such orphan GPCRs, little or no information may be known regarding their regulation, expression pattern, physiologic function, or role in pathophysiology. A large number of orphan receptors with no currently known endogenous agonists are detectable in the heart and other cardiovascular tissues and cells (Tang and Insel, Trends Cardiovasc. Med. 14:94-99, 2004; Hakak et al., FEBS Lett. 550:11-17, 2003; Katugampola and Davenport, Trends Pharmacol. Sci. 24:30-35, 2003).

GPCRs expressed in the heart are attractive targets for the development of drugs for the treatment of cardiac and other cardiovascular disorders. See, e.g., U.S. patent applications no. 2007/0232519; 2007/0231792; 2007/0224127; and 2005/0238579. See also, e.g., PCT patent applications no. WO 2004/013285; WO 2004/040000, and WO 2005/003786, which are incorporated herein in their entirety.

Patent documents related to GPCRs. Published patents and patent applications related to GPCRs are the following, which are incorporated herein by reference in their entirety include: US patent application nos. 20070224127 and 20070275410.

### SUMMARY OF THE INVENTION

The orphan type-1 GPCR, GPR22 (20RH), is exclusively expressed in the brain and heart in adults and plays a role in cardiac function (the terms 20RH and GPR22 are used interchangeably herein). We have found that down-regulation of the GPR22-mediated signal has a positive impact on cardiac morphology and function. Accordingly, the present invention includes methods of screening substances in vitro or in vivo in order to identify candidate substances, including but not limited to small molecule antagonists of GPR22G-protein coupled receptor activity, that improve cardiac morphology and function and affect patient survival. The present invention also includes methods of treatment.

Due to the enriched expression of GPR22 to the adult brain and heart, repression of GPR22 expression or activity, is expected to be benefitial for neural or behavioral disorders. Reduction of GPR22 activity or expression, will be also beneficial as a protective strategy to neuronal degenerative disease.

Accordingly, the present invention provides methods of identifying a candidate substance that reduces GPR22 activity in a cell comprising: (a) providing a cell comprising a level of GPR22 activity; (b) contacting the cell with the candidate substance; (c) determining whether the candidate substance reduces said level of GPR22 activity.

According to one embodiment, the candidate substance is an antagonist of GPR22G-protein coupled receptor activity.

According to another embodiment, the candidate substance reduces levels of GPR22 polypeptide in the cell. According to one such embodiment, the candidate substance is selected from the group consisting of an antisense polynucleotide, a ribozyme, and an siRNA molecule.

According to one embodiment, the cell is a cardiomyocyte cell, and the method comprises determining whether the candidate substance improves survival of the cardiomyocyte cell. According to one such embodiment, the method comprises contacting the cardiomyocyte cell with the candidate substance in vitro. According to another such embodiment, the method comprises determining whether the candidate substance improves survival of the cardiomyocyte cell by measuring apoptosis of the cardiomyocyte cell.

According to one embodiment, the cell is a brain cell, and the method comprises determining whether the candidate substance improves survival of the brain cell. According to one such embodiment, the method comprises contacting the brain cell with the candidate substance in vitro. According to another such embodiment, the method comprises determining whether the candidate substance improves survival of the brain cell by measuring apoptosis of the brain cell.

According to one embodiment, the method comprises administering the candidate substance to an animal comprising the cell, including, for example, an animal is selected from the group consisting of a mouse, rat, and pig, and preferably a mouse or rat. According to one such embodiment, the animal is a non-human animal comprising a gene encoding a polypeptide comprising a human GPR22 polypeptide or a fragment thereof having GPCR activity that is expressed in the cell (i.e., a transgenic animal). According to another such embodiment, the animal has impaired cardiovascular function resulting from a cardiovascular disease, disorder, or injury, including but not limited to one or more selected from the group consisting of ischemic heart disease (including but not limited to myocardial infarction), post-myocardial infarction remodeling, and congestive heart failure. According to another such embodiment, the animal is a surgical model of ischemic heart disease or heart failure. According to another such embodiment, the animal has impaired neurological function resulting from a disease, disorder, or injury of the brain.

The present invention also provides methods of preventing or treating a disease, disorder or injury of the heart or brain in an individual in need thereof, the method comprising administering to the individual a therapeutically effective amount of a substance that reduces GPR22 activity.

According to one embodiment, the disease, disorder or injury is a disease, disorder or injury of the heart. According to one such embodiment, the disease, disorder or injury is characterized by reduced cardiac output or increased venous pressure. According to another such embodiment, the disease, disorder or injury is selected from the group consisting of ischemic heart disease, post-myocardial infarction remodeling, and congestive heart failure.

According to another embodiment, the disease, disorder or injury is a disease, disorder or injury of the brain.

According to one embodiment, the individual is selected from the group consisting of a horse, cow, sheep, pig, cat, dog, rabbit, mouse, rat, non-human primate or human.

The present invention also provides compositions comprising a polynucleotide selected from the group consisting of an expression vector comprising an GPR22 promoter operably linked to a sequence that, when expressed, reduces GPR22 gene expression in a cell; an siRNA that reduces GPR22 gene expression; an antisense polynucleotide that reduces GPR22 gene expression; and a ribozyme that reduces GPR22 gene expression; wherein the composition is therapeutically effective in treating a disease, disorder or condition of heart or brain of an individual in need thereof. According to one such embodiment, the composition comprises the expression vector wherein the sequence encodes a polynucleotide selected from the group consisting of an siRNA, an antisense polynucleotide, and a ribozyme. According to another such embodiment, the composition comprises a carrier. According to another such embodiment, the composition is therapeutically effective in treating a disease, disorder or condition of the heart of the individual in need thereof. According to another such embodiment, the composition is therapeutically effective in treating a disease, disorder or condition of the brain of the individual in need thereof.

The present invention also provides methods of treating a disease, disorder or injury of the heart or brain in a patient in need of such treatment comprising administering to the patient an effective amount of such compositions. According to one such embodiment, the disease, disorder or injury is a disease, disorder or injury of the heart. According to another such embodiment, the disease, disorder or injury is a disease, disorder or injury of the brain.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE FIGURES

FIG. 1 shows the nucleotide sequence of the gene (genomic DNA) encoding human GPR22.

FIG. 2 shows the nucleotide sequence of the mRNA encoding human GPR22.

FIG. 3 shows the amino acid sequence of human GPR22.

FIG. 4A shows a diagram of the fldnRAR403 transgene.

FIG. 4B shows the results of an in vitro test of the fldnRAR403 construct, Cotransfection conditions included: (1) co-transfection of pBRE (RAR response element reporter plasmid); (2) double transfection of pBRE plus fldnRAR403 plasmid, which still showed responsiveness to treatment with at-retinoic acid (10⁻⁷ M), due to the absence of Cre expression; and (3) triple contransfection of pBRE, fldnRAR403 and a Cre-recombinase expression plasmid, showing inhibition of activation of the reporter plasmid as a result of dnRAR403 expression.

FIG. 5 shows a Kaplan Meier Survival curve of wild-type mice and transgenic fRAR403:MLC2v-Cre mice. Ten week old mice had enlarged left and right atria and showed symptoms of heart failure.

FIGS. 6A and B show that the average number of myocytes was increased in RAR403 over-expresser mice compared to wild-type mice at 2 and 6 weeks of age (p<0.05).

FIG. 7A shows hemodynamic measurements taken in 5 week old mice, indicating normal response to beta-adrenergic stimulation.

FIG. 7B shows hemodynamic evaluation of ten week old mice. RAR403:MLC2vCre transgenic mice showed a significantly blunted response to increasing doses of beta-adrenergic stimulation with Dobutamine. Ten week old mutant mice also displayed significant systolic dysfunction in M-mode echocardiograms (not shown).

FIG. 8 shows a representation of a survival study performed on eight animals of identical genetic background from genotypes, including wild-type (wt), ventricular dominant negative RAR (fRAR403:MLC2vCre) and ventricular double mutant for dominant negative RAR and 20RH (fRAR403:f20RH:MLC2vCre).

### DETAILED DESCRIPTION OF THE INVENTION

### Definitions and Methods

The following definitions and methods are provided to better define the present invention and to guide those of ordinary skill in the art in the practice of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR 1.822 is used. The standard one- and three-letter nomenclature for amino acid residues is used.

### Polynucleotides

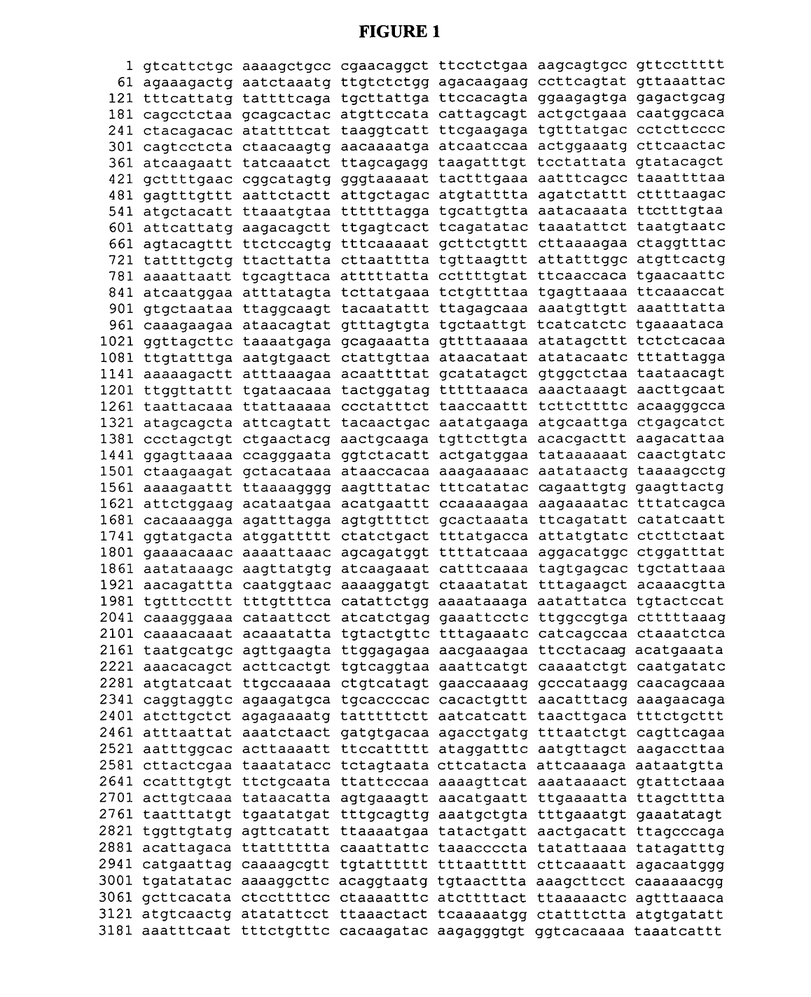

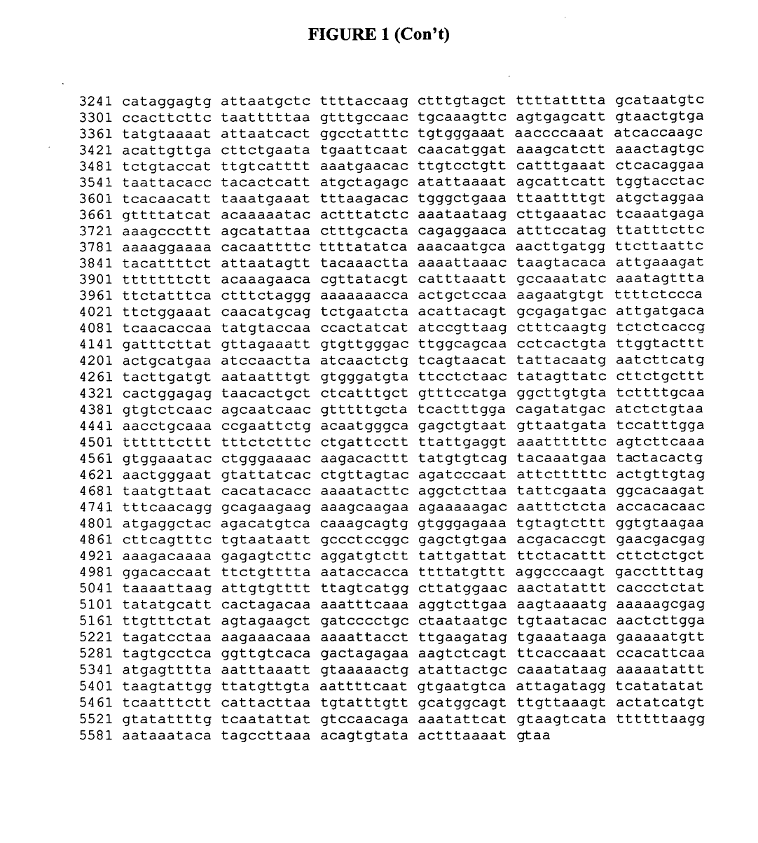

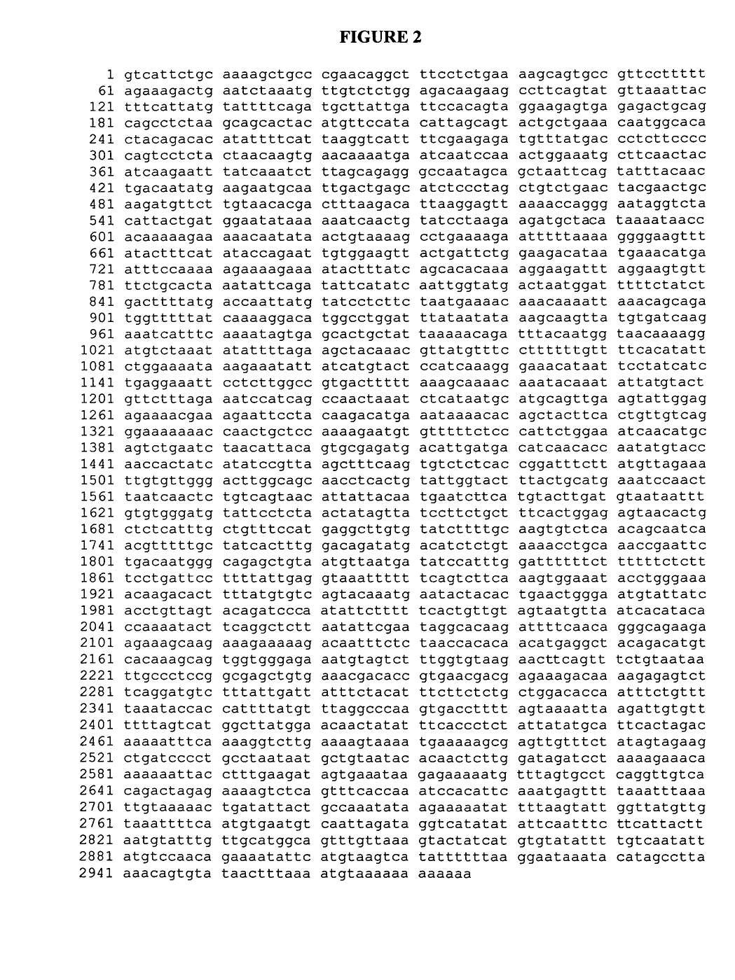

As used herein, the term “GPR22 polynucleotide (or probe or primer)” refers to a polynucleotide (or probe or primer) that encodes a GPR22 polypeptide or fragment thereof from any organism, including, without limitation, human GPR22 genomic DNA (FIG. 1), human GPR22 cDNA (FIG. 2), and mRNA, for example. These human polynucleotide sequences are available from the NCBI nucleotide sequence databases. GPR22 polynucleotides have also been identified in non-human organisms, including, but not limited to the following (genomic DNA sequences with GeneID reference numbers from the NCBI nucleotide sequence database): rhesus monkey (Macaca mulatta; GeneID: 699492); chimpanzee (Pan troglodytes; GenID: 744517); dog (Canis lupusfamiliaris; GeneID:607459); domestic cow (Bos taurus; Gene ID: 783685); mouse (Mus musculus; GeneID: 73010; Norway rat (Rattus norvegicus; GeneID: 298944); chicken (Gallus gallus; GeneID: 769379); and zebrafish (Danio rerio; GeneID: 607459). GPR22 is a member of the G-protein coupled receptor 1 family that is expressed in the heart and brain and that is involved in cardiac function, as discussed herein.

As used herein, the term “GPR22 polynucleotide” refers to native or wild-type GPR22 mRNA, the corresponding cDNA, including but not limited to the protein-coding region thereof. Also encompassed by the term “GPR22 polynucleotides” are, for example: fragments or portions of the GPR22 mRNA or cDNA; fragments that encode antigenic determinants of GPR22 (e.g., those that elicit antibodies that bind selectively to a GPR22 polypeptide); probes and primers that hybridize selectively to GPR22 polynucleotides; etc. Also included are mutated or variant polynucleotides that include one or more nucleotide insertions, deletions, or substitutions from the wild-type GPR22 sequence, but that, for example: retain the ability to bind selectively to GPR22 polynucleotides; encode a polypeptide that includes a GPR22 antigenic determinant; encode a polypeptide having GPR22 activity; etc.

As used herein, the term “hybridizes selectively” refers to binding of a probe, primer or other polynucleotide, under stringent hybridization conditions, to a target polynucleotide, such as a native, or wild-type, GPR22 mRNA or cDNA, to a substantially higher degree than to other polynucleotides. Probes and primers that hybridize selectively to GPR22 polynucleotides include sequences that are unique to a native GPR22 polynucleotide, i.e., are not found elsewhere in the genome of a particular organism. In particular, a probe that “hybridizes selectively” to GPR22 does not hybridize substantially to sequences other than a native GPR22 polynucleotide under stringent hybridization conditions and therefore can be used to distinguish a GPR22 polynucleotide (e.g., a GPR22 mRNA) from another polynucleotide. Similarly, a primer that “hybridizes selectively” to GPR22, when used in an amplification reaction such as PCR, results in amplification of a GPR22 polynucleotide without resulting in substantial amplification of other polynucleotide sequences under suitable amplification conditions. Thus, all or substantially all of a GPR22-selective probe or primer hybridizes to the target GPR22 polynucleotide under suitable conditions, as can be determined given the sensitivity of a particular procedure. Similarly, as used herein, the term “selective for” in reference to a polynucleotide, indicates that the polynucleotide hybridizes selectively to a target polynucleotide.

Similarly, a probe or primer that includes a sequence that is unique to GPR22, hybridizes selectively to GPR22. In particular, a probe that hybridizes selectively to GPR22 does not hybridize substantially to other polynucleotide sequences under stringent hybridization conditions and therefore can be used to distinguish a GPR22 polynucleotide (e.g., a GPR22 mRNA) from other polynucleotides. Similarly, a primer that hybridizes selectively to a GPR22 polynucleotide, when used in an amplification reaction such as PCR, results in amplification of the GPR22 polynucleotide without resulting in substantial amplification of other polynucleotides. Thus, all or substantially all of a GPR22-selective probe or primer hybridizes to the target GPR22 polynucleotide, as can be determined given the sensitivity of a particular procedure.

As used herein, the terms “wild-type” or “native” in reference to a polynucleotide are used interchangeably to refer to a polynucleotide that has 100% sequence identity with a reference polynucleotide that can be found in a cell or organism, or a fragment thereof.

Polynucleotide (e.g., DNA or RNA) sequences may be determined by sequencing a polynucleotide molecule using an automated DNA sequencer. A polynucleotide sequence determined by this automated approach can contain some errors. The actual sequence can be confirmed by resequencing the polynucleotide by automated means or by manual sequencing methods well known in the art.

Unless otherwise indicated, each “nucleotide sequence” set forth herein is presented as a sequence of deoxyribonucleotides (abbreviated A, G, C and T). However, the term “nucleotide sequence” of a DNA molecule as used herein refers to a sequence of deoxyribonucleotides, and for an RNA molecule, the corresponding sequence of ribonucleotides (A, G, C and U) where each thymidine deoxynucleotide (T) in the specified deoxynucleotide sequence in is replaced by the ribonucleotide uridine (U).

By “isolated” polynucleotide is intended a polynucleotide that has been removed from its native environment For example, recombinant polynucleotides contained in a vector are considered isolated for the purposes of the present invention. Further examples of isolated polynucleotides include recombinant polynucleotides maintained in heterologous host cells or purified (partially or substantially) polynucleotides in solution. Isolated RNA molecules include in vivo or in vitro RNA transcripts of the DNA molecules of the present invention. Isolated polynucleotides according to the present invention further include such molecules produced synthetically.

Polynucleotides can be in the form of RNA, such as mRNA, or in the form of DNA, including, for instance, cDNA and genomic DNA. The DNA can be double-stranded or single-stranded. A single-stranded DNA or RNA can be a coding strand, also known as the sense strand, or it can be a non-coding strand, also referred to as the anti-sense strand. Polynucleotides can include non-naturally occurring nucleotide or ribonucleotide analogs.

The term “fragment” (of a polynucleotide) as used herein refers to polynucleotides that are part of a longer polynucleotide having a length of at least about 15, 20, 25, 30, 35, or 40 nucleotides (nt) in length, which are useful, for example, as probes and primers. Thus, for example, a fragment of GPR22 cDNA at least 20 nucleotides in length includes 20 or more contiguous bases from the nucleotide sequence of the GPR22 full-length cDNA (as shown in FIG. 2). Such DNA fragments may be generated by the use of automated DNA synthesizers or by restriction endonuclease cleavage or shearing (e.g., by sonication) a full-length GPR22 cDNA, for example.

Also encompassed by the present invention are isolated polynucleotides that hybridize under stringent hybridization conditions to a GPR22 polynucleotide such as, for example, a GPR22 transcript (i.e., mRNA). By “stringent hybridization conditions” is intended overnight incubation at 42° C. in a solution comprising: 50% formamide, 5×SSC (750 mM NaCl, 75 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Alternatively, stringent hybridizations are conditions used for performance of a polymerase chain reaction (PCR). Such hybridizing polynucleotides are useful diagnostically as a probe according to conventional DNA hybridization techniques or as primers for amplification of a target sequence by the polymerase chain reaction (PCR).

As used herein, the term “hybridizes (or binds) specifically” is used interchangeably with the term “hybridizes (or binds) selectively” means that most or substantially all hybridization of a probe or primer is to a particular polynucleotide in a sample under stringent hybridization conditions.

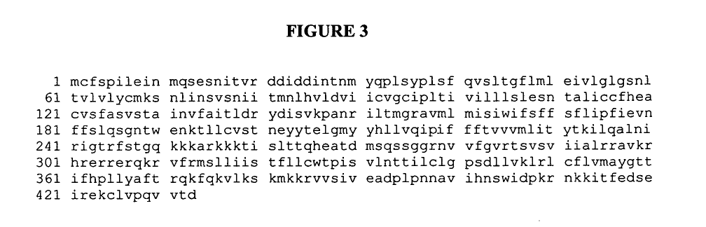

The present invention also provides polynucleotides that encode all or a portion of a polypeptide, e.g., a full-length GPR22 polypeptide (as shown in FIG. 3) or a portion thereof. Such protein-coding polynucleotides may include, but are not limited to, those sequences that encode the amino acid sequence of the particular polypeptide or fragment thereof and may also include together with additional, non-coding sequences, including for example, but not limited to introns and non-coding 5′ and 3′ sequences, such as the transcribed, non-translated sequences that play a role in transcription, mRNA processing—including splicing and polyadenylation signals, e.g., ribosome binding and stability of mRNA; an additional coding sequence which codes for additional amino acids, such as those which provide additional functionalities. In addition, the sequence encoding the polypeptide can be fused to a heterogeneous polypeptide or peptide sequence, such as, for example a marker sequence that facilitates purification of the fused polypeptide. One example of such a marker sequence is a hexa-histidine peptide, such as the tag provided in a pQE vector (Qiagen, Inc.). As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin (HA) protein, which has been described by Wilson et al., Cell 37:767 (1984).

The present invention further relates to variants of the native, or wild-type, polynucleotides of the present invention, which encode portions, analogs or derivatives of a GPR22 polypeptide. Variants can occur naturally, such as a natural allelic variant, i.e., one of several alternate forms of a gene occupying a given locus on a chromosome of an organism. Non-naturally occurring variants can be produced, e.g., using known mutagenesis techniques or by DNA synthesis. Such variants include those produced by nucleotide substitutions, deletions or additions. The substitutions, deletions or additions can involve one or more nucleotides. The variants can be altered in coding or non-coding regions or both. Alterations in the coding regions can produce conservative or non-conservative amino acid substitutions, deletions or additions. Also included are silent substitutions, additions and deletions, which do not alter the properties and activities of the GPR22 polypeptide or portions thereof.

Further embodiments of the invention include isolated polynucleotide molecules have, or comprise a sequence having, a high degree of sequence identity with a native, or wild type, GPR22 polynucleotide, for example, at least 90%, 95%, 96%, 97%, 98% or 99% identical thereto.

A polynucleotide is considered to have a nucleotide sequence at least, for example, 95% “identical” to a reference nucleotide sequence if it is identical to the reference sequence except that it includes up to five mutations (additions, deletions, or substitutions) per each 100 nucleotides of the reference nucleotide sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence. Nucleotide sequence identity may be determined conventionally using known computer programs such as the BESTFIT program (Wisconsin Sequence Analysis Package, Version 8 for Unix, Genetics Computer Group, University Research Park, 575 Science Drive, Madison, Wis. 53711. BESTFIT uses the local homology algorithm of Smith and Waterman, Adv. Appl. Math. 2:482-489 (1981), to find the best segment of homology between two sequences. When using BESTFIT or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

### Recombinant Constructs; Vectors and Host Cells

The present invention also provides recombinant polynucleotide constructs that comprise a GPR22 polynucleotide, including but not limited to vectors. The present invention also provides host cells comprising such vectors and the production of GPR22 polypeptides or fragments thereof by recombinant or synthetic techniques.

“Operably Linked”. A first nucleic-acid sequence is “operably linked” with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked DNA sequences are contiguous and, where necessary to join two protein coding regions, in reading frame.

“Recombinant”. A “recombinant” polynucleotide is made by an artificial combination of two otherwise separated segments of sequence, e.g., by chemical synthesis or by the manipulation of isolated segments of polynucleotides by genetic engineering techniques. Techniques for nucleic-acid manipulation are well-known (see, e.g., Sambrook et al., 1989, and Ausubel et al., 1992). Methods for chemical synthesis of polynucleotides are discussed, for example, in Beaucage and Carruthers, Tetra. Letts. 22:1859-1862, 1981, and Matteucci et al., J. Am. Chem. Soc. 103:3185, 1981. Chemical synthesis of polynucleotides can be performed, for example, on commercial automated oligonucleotide synthesizers.

Recombinant vectors are produced by standard recombinant techniques and may be introduced into host cells using well known techniques such as infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

Expression vectors include sequences that permit expression of a polypeptide encoded by a polynucleotide of interest in a suitable host cell. Such expression may be constitutive or non-constitutive, e.g., inducible by an environmental factor or a chemical inducer that is specific to a particular cell or tissue type, for example. Expression vectors include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

In expression vectors, a polynucleotide insert is operably linked to an appropriate promoter. The promoter may be a homologous promoter, i.e., a promoter or functional portion thereof, that is associated with the polynucleotide insert in nature, for example, a GPR22 promoter with a GPR22 protein-coding region. Alternatively, the promoter may be a heterologous promoter, i.e., a promoter or functional portion thereof, that is not associated with the polynucleotide insert in nature, for example, a bacterial promoter used for high-level protein expression in bacterial cells (or, for that matter, any promoter other than a GPR22 promoter) operably linked to a GPR22 protein coding region. The expression constructs will further contain sites for transcription initiation, termination and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vectors may include one or more selectable marker suitable for selection of a host cell into which such a vector has been introduced. Such markers include dihydrofolate reductase or neomycin resistance for eukaryotic cell culture and tetracycline or ampicillin resistance genes for culturing in E. coli and other bacteria. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture media and conditions for the above-described host cells are known in the art.

Bacterial promoters suitable include the E. coli lacI and lacZ promoters, the T3 and T7 promoters, the gpt promoter, the lambda PR and PL promoters and the trp promoter. Eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

A polypeptide of interest may be expressed in a modified form, such as a fusion protein, and may include not only secretion signals but also additional heterologous functional regions. For instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

An expressed polypeptide of interest can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Polypeptides

As used herein, the phrase “a GPR22 polypeptide” refers to a polypeptide at least 10, 11, 12, 12, 14, 15, 20, 30, 40, 49, 50, 100 or more amino acid residues in length and have a high degree of sequence identity with the full-length native, or wild-type, GPR22 polypeptide or a fragment thereof. Included are variant forms of GPR22 polypeptides that include deletions, insertions or substitutions of one or more amino acid residues in a native GPR22 polypeptide sequence, including without limitation polypeptides that exhibit activity similar, but not necessarily identical, to an activity of the full-length native, or wild-type, GPR22 polypeptide or fragment thereof as measured in a relevant biological assay.

As used herein, the terms “wild-type” or “native” in reference to a peptide or polypeptide are used interchangeably to refer to a polypeptide that has 100% sequence identity with a reference polypeptide that can be found in a cell or organism, or a fragment thereof.

As used herein, the terms “peptide” and “oligopeptide” are considered synonymous and, as used herein, each term refers to a chain of at least two amino acids coupled by peptidyl linkages. As used herein, the terms “polypeptide” and “protein” are considered synonymous and each term refers to a chain of more than about ten amino acid residues. All oligopeptide and polypeptide formulas or sequences herein are written from left to right and in the direction from amino terminus to carboxy terminus.

As used herein, the term “isolated” polypeptide or protein refers to a polypeptide or protein removed from its native environment. For example, recombinantly produced polypeptides and proteins expressed in host cells are considered isolated for purposes of the invention as are native or recombinant polypeptides and proteins which have been substantially purified by any suitable technique.

As used herein, the term “binds selectively” is interchangeable with the term “binds specifically, and, when used in reference to a GPR22 polypeptide, refers to binding of an antibody, ligand, receptor, substrate, or other binding agent to the target GPR22 polypeptide to a substantially higher degree than to other polypeptides. According to some embodiments, all or substantially all binding of an antibody or other binding agent is to the target GPR22 polynucleotide, as can be determined given the sensitivity of a particular procedure. An antibody, ligand, receptor, substrate or other binding agent is said to be “selective for” or specific for” a polypeptide or other target molecule if it binds selectively to the target molecule.

The amino acid sequence of a GPR22 polypeptide or peptide can be varied without significant effect on the structure or function of the protein. In general, it is possible to replace residues which contribute to the tertiary structure of the polypeptide or peptide, provided that residues performing a similar function are used. In other instances, the type of residue may be completely unimportant if the alteration occurs at a non-critical region of the protein.

Thus, the invention further includes variations of GPR22 polypeptide or peptide that show substantial GPR22 activity. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Guidance concerning which amino acid changes are likely to be phenotypically silent (i.e., are not likely to have a significant deleterious effect on a function) can be found, for example, in Bowie et al., Science 247:1306-1310, 1990.

Thus, a fragment, derivative or analog of a native, or wild-type GPR22 polypeptide, may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence that is employed for purification of the mature polypeptide or a proprotein sequence.

Charged amino acids may be substituted with another charged amino acid. Charged amino acids may also be substituted with neutral or negatively charged amino acids, resulting in proteins with reduced positive charge. The prevention of aggregation is highly desirable to avoid a loss of activity and increased immunogenicity (Pinckard et al., Clin Exp. Immunol. 2:331-340, 1967; Robbins et al., Diabetes 36:838-845, 1987; Cleland et al., Crit. Rev. Therapeutic Drug Carrier Systems 10:307-377, 1993).

The replacement of amino acids can also change the selectivity of protein binding to cell surface receptors. Ostade et al., Nature 361:266-268, 1993, describes certain mutations resulting in selective binding of TNF-α to only one of the two known types of TNF receptors, for example.

It is well known in the art that one or more amino acids in a native sequence can be substituted with other amino acid(s), the charge and polarity of which are similar to that of the native amino acid, i.e., a conservative amino acid substitution, resulting in a silent change. Conservative substitutes for an amino acid within the native polypeptide sequence can be selected from other members of the class to which the amino acid belongs. Amino acids can be divided into the following four groups: (1) acidic amino acids, (2) basic amino acids, (3) neutral polar amino acids, and (4) neutral, nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine. Conservative amino acid substitution within the native polypeptide sequence can be made by replacing one amino acid from within one of these groups with another amino acid from within the same group. In one aspect, biologically functional equivalents of the proteins or fragments thereof of the present invention can have ten or fewer, seven or fewer, five or fewer, four or fewer, three or fewer, two, or one conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, a protein with like properties can still be obtained. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157:105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, J. Mol. Biol. 157:105-132, 1982); these are: isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (4.5). In making such changes, the substitution of amino acids whose hydropathic indices may be within ±2, or ±1, or within ±0.5.

It is also understood in the art that the substitution of like amino acids can be made effectively on the basis of hydrophilicity. U.S. Pat. No. 4,554,101 states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0.+−0.1), glutamate (+3.0.+−0.1), serine (+0.3), asparagine (+0.2), glutamine (+0.2), glycine (0), threonine (−0.4), proline (−0.5.+−0.1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4). In making changes to a native polypeptide or peptide sequence, the substitution of amino acids whose hydrophilicity values may be within ±2, or within ±1, or within ±0.5.

Of course, the number of amino acid substitutions a skilled artisan would make depends on many factors, including those described above. Generally speaking, the number of substitutions for any given GPR22 polypeptide will not be more than 50, 40, 30, 20, 10, 5, 3, or 2.

Amino acids in the GPR22 protein of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as in vitro or in vivo ligand or receptor binding or other characteristic biological activities. Sites that are critical for ligand-receptor binding can also be determined by structural analysis such as crystallization, nuclear magnetic resonance or photoaffinity labeling (Smith et al., J. Mol. Biol. 224:899-904, 1992; de Vos et al. Science 255:306-312, 1992).

The polypeptides and peptides of the present invention include native, or wild-type polypeptides and peptides, and polypeptides or peptide variants that are at least 75%, 80%, 85%, 90%, 95%, 96%, 97%, 98% or 99% identical to (or have such a degree of identity with) the native GPR22 polypeptide and fragments thereof.

By a polypeptide having an amino acid sequence at least, for example, 95% “identical” to a reference amino acid sequence is intended that the amino acid sequence of the polypeptide is identical to the reference sequence except that the polypeptide sequence may include up to five amino acid alterations per each 100 amino acids of the reference amino acid sequence of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide has a particular degree of amino acid sequence identity when compared to a reference polypeptide can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In another embodiment of the present invention, there are provided fragments of the polypeptides described herein, including but not limited to GPR22 fragments that retain G-protein coupled receptor activity. The polypeptide fragments of the present invention can be used for numerous purposes, for example, to elicit antibody production in a mammal, as molecular weight markers on SDS-PAGE gels or on molecular sieve gel filtration columns using methods well known to those of skill in the art, etc.

Polypeptides of the present invention can be used to raise polyclonal and monoclonal antibodies, which are useful in diagnostic assays for detecting GPR22 expression or for other purposes. Further, such polypeptides can be used in the yeast two-hybrid system to “capture” binding proteins (Fields and Song, Nature 340:245-246, 1989).

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002, 1984).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science 219:660-666, 1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective (Sutcliffe et al., supra, at 661).

Antigenic epitope-bearing peptides and polypeptides of the invention are therefore useful to raise antibodies, including monoclonal antibodies, which bind selectively to a polypeptide of the invention. Thus, a high proportion of hybridomas obtained by fusion of spleen cells from donors immunized with an antigen epitope-bearing peptide generally secrete antibody reactive with the native protein (Sutcliffe et al., supra, at 663). The antibodies raised by antigenic epitope-bearing peptides or polypeptides are useful to detect the mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for example, Wilson et al., Cell 37:767-778, 1984). The anti-peptide antibodies of the invention also are useful for protein purification, e.g., by adsorption chromatography using known methods.

Antigenic epitope-bearing peptides and polypeptides of the invention designed according to the above guidelines may contain a sequence of at least 7, 8, 9, 10, 11, 12, 13, 14, 15, 20 or 30 or more amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein.

The amino acid sequence of the epitope-bearing peptide may be selected to provide substantial solubility in aqueous solvents (i.e., sequences including relatively hydrophilic residues and highly hydrophobic sequences may be avoided).

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies. Epitope-bearing peptides also may be synthesized using known methods of chemical synthesis. For instance, a simple method has been described for synthesis of large numbers of peptides, such as 10-20 mg of 248 different 13 residue peptides representing single amino acid variants of a segment of the HA1 polypeptide which were prepared and characterized (by binding studies employing an enzyme-linked immunosorbent assay [ELISA]) in less than four weeks (Houghten, Proc. Natl. Acad. Sci. USA 82:5131-5135, 1985; and U.S. Pat. No. 4,631,211). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously.

Epitope-bearing peptides and polypeptides of the invention are used to induce antibodies according to methods well known in the art. See, for instance, Sutcliffe et al., supra; Wilson et al., supra; Chow et al., Proc. Natl. Acad. Sci. USA 82:910-914; and Bittle et al., J. Gen. Virol. 66:2347-2354, 1985). Generally, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine may be coupled to carrier using a linker such as m-maleimidobenzoyl-N-hydroxysuccinimide ester (MBS), while other peptides may be coupled to carrier using a more general linking agent such as glutaraldehyde. Animals such as rabbits, rats and mice are immunized with either free or carrier-coupled peptides, for instance, by intraperitoneal and/or intradermal injection of emulsions containing about 100 μg peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Immunogenic epitope-bearing peptides of the invention, i.e., those parts of a protein that elicit an antibody response when the whole protein is the immunogen, are identified according to methods known in the art. For instance, Geysen et al. (1984), supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 further describes this method of identifying a peptide bearing an immunogenic epitope of a desired protein.

U.S. Pat. No. 5,194,392 describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 describes a method of detecting or determining a sequence of monomers which is a topographical equivalent of a ligand which is complementary to the ligand binding site of a particular receptor of interest. Similarly, U.S. Pat. No. 5,480,971 discloses linear C₁₋₇-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

As one of skill in the art will appreciate, polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86, 1988). Fusion proteins that have a disulfide-linked dimeric structure due to the IgG part can also be more efficient in binding and neutralizing other molecules than the monomeric GPR22 protein or protein fragment alone (Fountoulakis et al., J. Biochem. 270:3958-3964, 1995).

Antibodies that Inhibit GPR22 Activity

Antibodies can be used as modulators of the activity of a particular protein, such as, for example GPR22. For example, antibodies can bind to GPR22 so as to reduce its activity, such as, for example, by sterically hindering such binding or altering the conformation of GPR22. Both monoclonal and polyclonal antibodies (Ab) directed against a particular polypeptide, such as a GPR22 polypeptide, and antibody fragments such as Fab, F(ab)₂, Fv and scFv can be used to block the action of a particular protein, such as GPR22.

### Diagnostic Methods

There are various methods for detecting the presence of GPR22 polynucleotides (for example, GPR22 mRNA) or polypeptides in a sample, such as a biological sample from an individual; for quantitating GPR22 polynucleotides or polypeptides in a sample; etc.

In one such methods, a measurement of GPR22 polypeptide or polynucleotide is compared to a “reference.” Depending on the embodiment of the invention, such a reference can include a measurement or ratio in a control sample; a standard value obtained by measurements of a population of individuals; a baseline value determined for the same individual at an earlier timepoint, e.g., before commencing a course of treatment; or any other suitable reference used for similar methods.

As used herein, the term “individual” or “patient” refers to a mammal, including, but not limited to, a mouse, rat, rabbit, cat, dog, monkey, ape, human, or other mammal.

By “biological sample” is intended any biological sample obtained from an individual, including but not limited to, a body fluid, cell, tissue, tissue culture, or other source that contains GPR22 protein or mRNA. Methods for obtaining tissue biopsies and other biological samples from mammals are well known in the art.

“Modulate”. As used herein, the term “modulate” means to detectably change the expression of an expressible polynucleotide sequence in any detectable fashion, including but not limited to increasing or decreasing the level of expression, the timing of expression, the cell, tissue, organ or other specificity of expression, or any other aspect of gene expression. A modulation of gene expression may be detected by any know means, including, but not limited to, detecting a change in the level of mRNA transcription, of protein encoded by the polynucleotide, of enzymatic activity corresponding to an encoded protein, etc.

Detection of mRNA. Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding GPR22 are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al., Cell 63:303-312, 1990). Briefly, total RNA is prepared from a biological sample as described above. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. GPR22 cDNA labeled according to any appropriate method (such as a ³²P-multiprimed DNA labeling system is used as probe. After hybridization overnight, the filter is washed and exposed to x-ray film. cDNA for use as probe according to the present invention is described in the sections above.

S1 mapping can be performed as described in Fujita et al., Cell 49:357-367, 1987). To prepare probe DNA for use in S1 mapping, the sense strand of above-described cDNA is used as a template to synthesize labeled antisense DNA. The antisense DNA can then be digested using an appropriate restriction endonuclease to generate further DNA probes of a desired length. Such antisense probes are useful for visualizing protected bands corresponding to the target mRNA (i.e., mRNA encoding GPR22). Northern blot analysis can be performed as described above.

According to one embodiment, levels of mRNA encoding GPR22 are assayed using a polynucleotide amplification method, including but not limited to a polymerase chain reaction (PCR). One PCR method that is useful in the practice of the present invention is the RT-PCR method described in Makino et al., Technique 2:295-301, 1990), for example. By this method, the radioactivity of the DNA products of the amplification, i.e., the “amplification products” or “amplicons,” in the polyacrylamide gel bands is linearly related to the initial concentration of the target mRNA. Briefly, this method involves adding total RNA isolated from a biological sample in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands (corresponding to the mRNA encoding GPR22 is quantified using an imaging analyzer. RT and PCR reaction ingredients and conditions, reagent and gel concentrations, and labeling methods are well known in the art.

According to one embodiment of an amplification method of the invention, primers are employed that selectively amplify a GPR22 polynucleotide in a sample, for example, a primer pair including at least one primer that selectively hybridizes to GPR22 mRNA (e.g., that includes sequences from the region of the GPR22 mRNA that encodes the GPR22 polypeptide. The second primer can include any sequence from the target GPR22 polynucleotide, whether such a sequence is unique to GPR22. This embodiment is useful for amplifying only a GPR22 transcript (mRNA) in a sample, for example.

According to another embodiment of the invention, primers are employed that selectively amplify a GPR22 polynucleotide, for example, a primer pair that includes at least one primer that selectively hybridizes to GPR22 mRNA (e.g., that includes sequences from exon 4a. The second primer can include any sequence from the target GPR22 polynucleotide, whether such a sequence is unique to GPR22. This embodiment is useful for amplifying only a GPR22 transcript (mRNA) in a sample, for example.

The skilled artisan will be able to produce additional primers, primer pairs, and sets of primers for PCR and other amplification methods based on the sequences taught herein.

One embodiment of the present invention is a kit that includes primers useful for amplification methods according to the present invention. Such kits also include suitable packaging, instructions for use, or both.

Another PCR method useful for detecting the presence of and/or quantitating GPR22 mRNA and protein in a biological sample is through the use of “bio-barcode” nanoparticles. For detection and/or quantitation of proteins, for example, two types of capture particles are employed: one is a micro-size magnetic particle bearing an antibody selective for a target protein, and the other is a nanoparticle with attached antibodies selective for the same protein. The nanoparticle also carries a large number (e.g., ˜100) of unique, covalently attached oligonucleotides that are bound by hybridization to complementary oligonucleotides. The latter are the “bio-barcodes” that serve as markers for a selected protein. Because the nanoparticle probe carries many oligonucleotides per bound protein, there is substantial amplification, relative to protein. There is a second amplification of signal in a silver enhancement step. The result is 5-6 orders of magnitude greater sensitivity for proteins than ELISA-based assays, by detecting tens to hundreds of molecules. See, e.g., U.S. Pat. No. 6,974,669. See also, e.g., Stoeva et al., J. Am. Chem. Soc. 128:8378-8379, 2006, for an example of detection of protein cancer markers with bio-barcoded nanoparticle probes. The bio-barcode method can also be used for detecting and/or quantitating mRNA and other polynucleotides in a sample (Huber et al., Nucl. Acids Res. 32:e137, 2004; Cheng et al., Curr. Opin. Chem. Biol. 10:11-19, 2006; Thaxton et al., Clin. Chim. Acta 363:120-126, 2006; U.S. Pat. No. 6,974,669).

Detection of polypeptides. Assaying the presence of, or quantitating, GPR22 polypeptide in a biological sample can occur using any art-known method.

Antibody-based techniques are useful for detecting the presence of and/or quantitating GPR22 levels in a biological sample. For example, GPR22 expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of GPR22 for Western-blot or dot/slot assay (Jalkanen et al., J. Cell. Biol. 101:976-985, 1985; Jalkanen et al., J. Cell. Biol. 105:3087-3096, 1987). In this technique, which is based on the use of cationic solid phases, quantitation of GPR22 can be accomplished using isolated GPR22 as a standard. This technique can also be applied to body fluids. With these samples, a molar concentration of GPR22 will aid to set standard values of GPR22 content for different tissues. The normal appearance of GPR22 amounts can then be set using values from healthy individuals, which can be compared to those obtained from a test subject.

Other antibody-based methods useful for detecting GPR22 levels include immunoassays, such as the enzyme linked immunosorbent assay (ELISA), the radioimmunoassay (RIA), and the “bio-barcode” assays described above. For example, GPR22-selective monoclonal antibodies can be used both as an immunoadsorbent and as an enzyme-labeled probe to detect and quantify the GPR22. The amount of GPR22 present in the sample can be calculated by reference to the amount present in a standard preparation using a linear regression computer algorithm. Such an ELISA for detecting a tumor antigen is described in lacobelli et al., Breast Cancer Research and Treatment 11: 19-30, 1988. In another ELISA assay, two distinct selective monoclonal antibodies can be used to detect GPR22 in a body fluid. In this assay, one of the antibodies is used as the immunoadsorbent and the other as the enzyme-labeled probe.

The above techniques may be conducted essentially as a “one-step” or “two-step” assay. The “one-step” assay involves contacting GPR22 with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample.

Suitable enzyme labels include, for example, those from the oxidase group, which catalyze the production of hydrogen peroxide by reacting with substrate. Glucose oxidase, for example, has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulfur (³⁵S), tritium (³H), indium (¹¹²In), and technetium (⁹⁹Tc), and fluorescent labels, such as fluorescein and rhodamine, and biotin.

In addition to assaying GPR22 levels in a biological sample obtained from an individual, GPR22 can also be detected in vivo by imaging. Antibody labels or markers for in vivo imaging of GPR22 include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A GPR22-selective antibody or antibody fragment which has been labeled with an appropriate detectable imaging moiety, such as a radioisotope (for example, ¹³¹I, ¹¹²In, ⁹⁹ mTc), a radio-opaque substance, or a material detectable by nuclear magnetic resonance, is introduced (for example, parenterally, subcutaneously or intraperitoneally) into the mammal to be examined for a disorder. It will be understood in the art that the size of the subject and the imaging system used will determine the quantity of imaging moieties needed to produce diagnostic images. In the case of a radioisotope moiety, for a human subject, the quantity of radioactivity injected will normally range from about 5 to 20 millicuries of ⁹⁹ mTc. The labeled antibody or antibody fragment will then preferentially accumulate at the location of cells which contain GPR22. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, eds., Masson Publishing Inc., 1982).

GPR22-selective antibodies for use in the present invention can be raised against the intact GPR22 or an antigenic polypeptide fragment thereof, which may presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (or “fragment antibodies”) (such as, for example, Fab and F(ab′).sub.2 fragments) which are capable of selectively binding to GPR22. Fab and F(ab′).sub.2 fragments lack the Fc portion of intact antibody, clear more rapidly from the circulation, and may have less non-specific tissue binding of an intact antibody (Wahl et al., J. Nucl. Med. 24:316-325, 1983).

The antibodies of the present invention may be prepared by any of a variety of methods. For example, cells expressing the GPR22 or an antigenic fragment thereof can be administered to an animal in order to induce the production of sera containing polyclonal antibodies. In one method, a preparation of GPR22 protein is prepared and purified as described above to render it substantially free of natural contaminants. Such a preparation is then introduced into an animal in order to produce polyclonal antisera of greater specific activity.

The antibodies of the present invention include monoclonal antibodies (or GPR22 binding fragments thereof). Such monoclonal antibodies can be prepared using hybridoma technology (Colligan, Current Protocols in Immunology, Wiley Interscience, New York (1990-1996); Harlow & Lane, Antibodies: A Laboratory Manual, Cold Spring Harbor Press, Cold Spring Harbor, N.Y. (1988), Chapters 6-9, Current Protocols in Molecular Biology, Ausubel, infra, Chapter 11). In general, such procedures involve immunizing an animal (for example, a mouse or rabbit) with a GPR22 antigen or with a GPR22-expressing cell. Suitable cells can be recognized by their capacity to bind anti-GPR22 antibody. Such cells may be cultured in any suitable tissue culture medium, such as Earle's modified Eagle's medium supplemented with 10% fetal bovine serum (inactivated at about 56° C.), and supplemented with about 10 μg/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described by Wands et al., Gastroenterology 80:225-232, 1981); Harlow & Lane, infra, Chapter 7. The hybridoma cells obtained through such a selection are then assayed to identify clones which secrete antibodies capable of binding the GPR22 antigen.

Alternatively, additional antibodies capable of binding to the GPR22 antigen may be produced in a two-step procedure through the use of anti-idiotypic antibodies. Such a method makes use of the fact that antibodies are themselves antigens, and therefore it is possible to obtain an antibody which binds to a second antibody. In accordance with this method, GPR22-selective antibodies are used to immunize an animal, such as a mouse. The splenocytes of such an animal are then used to produce hybridoma cells, and the hybridoma cells are screened to identify clones which produce an antibody whose ability to bind to the GPR22-selective antibody can be blocked by the GPR22 antigen. Such antibodies comprise anti-idiotypic antibodies to the GPR22-selective antibody and can be used to immunize an animal to induce formation of further GPR22-selective antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, GPR22-binding fragments can be produced through recombinant DNA technology or protein synthesis.

Where in vivo imaging is used to detect enhanced levels of GPR22 for diagnosis in humans, one may use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202, 1985; Oi et al., BioTechniques 4:214, 1986; Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643, 1984; Neuberger et al., Nature 314:268, 1985.

Further suitable labels for the GPR22-selective antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ⁰⁹Pd, etc. ¹¹¹In has advantages where in vivo imaging is used since its avoids the problem of dehalogenation of the ¹²⁵I- or ¹³¹I-labeled monoclonal antibody by the liver. In addition, this radionucleotide has a more favorable gamma emission energy for imaging (Perkins et al., Eur. J. Nucl. Med. 10:296-301, 1985); Carasquillo et al., J. Nucl. Med. 28:281-287, 1987). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870, 1987).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include ¹⁵²Eu label, fluorescein, isothiocyanate, rhodamine, phycoerythrin, phycocyanin, allophycocyanin, o-phthaldehyde, and fluorescamine.

Examples of suitable toxin labels include diphtheria toxin, ricin, and cholera toxin. Examples of chemiluminescent labels include luminal, isoluminal, aromatic acridinium ester, imidazole, acridinium salt, oxalate ester, luciferin, luciferase, and aequorin.

Examples of nuclear magnetic resonance contrasting agents include heavy metal nuclei such as Gd, Mn, and Fe.

Typical techniques for binding the above-described labels to antibodies are provided by Kennedy et al. (Clin. Chim. Acta 70:1-31, 1976), and Schurs et al. (Clin. Chim. Acta 81:1-40, 1977). Coupling techniques mentioned in the latter are the glutaraldehyde method, the periodate method, the dimaleimide method, the m-maleimidobenzyl-N-hydroxy-succinimide ester method.

One use for diagnostic compositions and methods of the present invention is for the detection of the presence of GPR22 (or another condition marked by the up-regulation and/or increased expression of GPR22) in a patient. Another use is to determine the presence and level of GPR22 (mRNA or polypeptide) in a patient in order to monitor the efficacy of therapy directed toward treatment of a cardiovascular disorder, disease or injury. The diagnostic compositions and method of the present invention are also useful for determining the efficacy of therapeutic agents for treatment and prophylaxis of such cardiovascular disorders, diseases or injuries.

### Reducing Intracellular Levels of GPR22

Compositions and methods for reducing intracellular levels of GPR22 are useful for the practice of the present invention. The methods and compositions of the present invention can be used in relation to any type of heart, brain or other cell in which GPR22 is expressed. As used herein, the term “cell” or “cells” is intended to include not only single cells but also tissues and even whole organisms, as would be appropriate for the context.

“Inhibition of GPR22 therapy” refers to therapy based on the inhibition of GPR22 activity. For example, in certain embodiments of the present invention, inhibition of GPR22 therapy includes inhibiting the activity of GPR22 polypeptide in a cell (e.g., a heart or brain cell) in a patient in need of treatment. Inhibition of GPR22 therapy can be applied to any type of disease, condition or injury in which such therapy would be beneficial. In certain other embodiments, the invention relates to agents that inhibit GPR22 activity, including without limitation, G-protein coupled receptor activity.

Inhibition of molecules involved in GPR22 activation. In certain embodiments, the present invention contemplates inhibiting GPR22 in cells by inhibiting one or more of the molecules that are directly or indirectly involved in GPR22 activation, e.g., transcription factors that are involved in GPR22 expression.

Polynucleotides that reduce intracellular levels of GPR22. Therapeutic use of molecules that inhibit GPR22 activity include the delivery to cells of polynucleotides that reduce intracellular levels of GPR22, preferably without substantially reducing intracellular levels of other proteins.

In certain embodiments of the invention, GPR22 activity is inhibited through the use of antisense, ribozyme, RNAi, and other nucleic acid-related methods and compositions for inhibiting an GPR22 activity. Any of the nucleic acid therapies of the invention may be designed to target a nucleic acid sequence represented in an GPR22 nucleic acid. In certain embodiments, any of the nucleic acid therapies of the invention may be designed to target a nucleic acid sequence represented in a nucleic acid sequence of a molecule involved in the activation of GPR22.

RNA interference. The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an “siRNA” and includes small interfering RNA (siRNA), short hairpin RNA (shRNA), etc. and mimetics thereof (including but not limited to polynucleotides that include non-canonical nucleoside mimetics such as, for example, 2,4-diflulorotoluoyl ribonucleoside, among others; see, e.g., Xia et al., ACS Chem. Biol. 1:176-183, 2006)

Certain embodiments of the invention make use of materials and methods for reducting the expression of a GPR22 genes by means of RNAi. Additional embodiments of the invention make use of materials and methods for effecting knockdown of one or more genes involved in the activation of GPR22. RNAi is a process of sequence-specific post-transcriptional gene repression which can occur in eukaryotic cells. RNAi has been shown to be effective in reducing or eliminating the expression of genes in a number of different organisms including Caenorhabditiis elegans (see e.g., Fire et al., Nature 391:806-811, 1998), mouse eggs and embryos (Wianny et al., Nature Cell Biol. 2:70-75, 2000; Svoboda et al., Development 127:4147-4156, 2000), and cultured RAT-1 fibroblasts (Bahramina et al., Mol Cell Biol. 19:274-283, 1999), and appears to be an anciently evolved pathway available in eukaryotic plants and animals (Sharp, Genes Dev. 15:485-490, 2001). RNAi has proven to be an effective means of decreasing gene expression in a variety of cell types including HeLa cells, NIH/3T3 cells, COS cells, 293 cells and BHK-21 cells.

The double stranded oligonucleotides used to effect RNAi may be less than 30 base pairs in length, for example, comprising about 25, 24, 23, 22, 21, 20, 19, 18 or 17 base pairs of ribonucleic acid. Optionally the dsRNA oligonucleotides of the application may include 3′ overhang ends. dsRNAs may be synthesized chemically or produced in vitro or in vivo using appropriate expression vectors. Synthetic RNAs include RNAs that are chemically synthesized using methods known in the art (e.g., Expedite RNA phosphoramidites and thymidine phosphoramidite (Proligo, Germany). Synthetic oligonucleotides may be deprotected and gel-purified using methods known in the art (see e.g., Elbashir et al., Genes Dev. 15:188-200, 2001). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species may be designed to include a portion of nucleic acid sequence represented in a GPR22 nucleic acid. RNAi constructs of the invention further include RNAi constructs designed to include a portion of nucleic acid sequence represented in a gene involved in the activation of GPR22. Methods and compositions for designing appropriate oligonucleotides may be found, for example, in U.S. Pat. No. 6,251,588, the contents of which are incorporated herein by reference. Further compositions, methods and applications of RNAi technology are provided in U.S. Pat. Nos. 6,278,039, 5,723,750 and 5,244,805, which are incorporated herein by reference.

A gene is “targeted” by a siNA according to the invention when, for example, the siNA molecule selectively decreases or inhibits the expression of the gene. The phrase “selectively decrease or inhibit” as used herein encompasses siNAs that affects expression of one gene as well those that effect the expression of more than one gene. In cases where an siNA affects expression of more than one gene, the gene that is targeted is effected at least two times, three times, four times, five times, ten times, twenty five times, fifty times, or one hundred times as much as any other gene. Alternatively, a siNA targets a gene when the siNA hybridizes under stringent conditions to the gene transcript. siNAs can be tested either in vitro or in vivo for the ability to target a gene.

A short fragment of the target gene sequence (e.g., 19-40 nucleotides in length) is chosen as the sequence of the siNA of the invention. In one embodiment, the siNA is a siRNA. In such embodiments, the short fragment of target gene sequence is a fragment of the target gene mRNA. In preferred embodiments, the criteria for choosing a sequence fragment from the target gene mRNA to be a candidate siRNA molecule include: (1) a sequence from the target gene mRNA that is at least 50-100 nucleotides from the 5′ or 3′ end of the native mRNA molecule, (2) a sequence from the target gene mRNA that has a G/C content of between 30% and 70%, most preferably around 50%, (3) a sequence from the target gene mRNA that does not contain repetitive sequences (e.g., AAA, CCC, GGG, TTT, AAAA, CCCC, GGGG, TTTT), (4) a sequence from the target gene mRNA that is accessible in the mRNA, and (5) a sequence from the target gene mRNA that is unique to the target gene. The sequence fragment from the target gene mRNA may meet one or more of the criteria identified supra. In embodiments where a fragment of the target gene mRNA meets less than all of the criteria identified supra, the native sequence may be altered such that the siRNA conforms with more of the criteria than does the fragment of the target gene mRNA. In preferred embodiments, the siRNA has a G/C/ content below 60% and/or lacks repetitive sequences.

In some embodiments, each of the siNAs of the invention targets one gene. In one specific embodiment, the portion of the siNA that is complementary to the target region is perfectly complementary to the target region. In another specific embodiment, the portion of the siNA that is complementary to the target region is not perfectly complementary to the target region. siNA with insertions, deletions, and point mutations relative to the target sequence are also encompassed by the invention. Thus, sequence identity may calculated by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90%, 95%, or 99% sequence identity between the siNA and the portion of the target gene is preferred. Alternatively, the complementarity between the siNA and native RNA molecule may be defined functionally by hybridization. A siNA sequence of the invention is capable of hybridizing with a portion of the target gene transcript under stringent conditions (e.g., 400 mM NaCl, 40 mM PIPES pH 6.4, 1 mM EDTA, 50° C. or 70° C. hybridization for 12-16 hours; followed by washing). A siNA sequence of the invention can also be defined functionally by its ability to decrease or inhibit the expression of a target gene. The ability of a siNA to effect gene expression can be determined empirically either in vivo or in vitro.

In addition to siNAs which specifically target only one gene, degenerate siNA sequences may be used to target homologous regions of multiple genes. WO2005/045037 describes the design of siNA molecules to target such homologous sequences, for example by incorporating non-canonical base pairs, for example mismatches and/or wobble base pairs, that can provide additional target sequences. In instances where mismatches are identified, non-canonical base pairs (for example, mismatches and/or wobble bases) can be used to generate siNA molecules that target more than one gene sequence. In a non-limiting example, non-canonical base pairs such as UU and CC base pairs are used to generate siNA molecules that are capable of targeting sequences for differing targets that share sequence homology. As such, one advantage of using siNAs of the invention is that a single siNA can be designed to include nucleic acid sequence that is complementary to the nucleotide sequence that is conserved between homologous genes. In this approach, a single siNA can be used to inhibit expression of more than one gene instead of using more than one siNA molecule to target different genes.

In some embodiments of the invention, siNA molecules are double stranded. In one embodiment, double stranded siNA molecules comprise blunt ends. In another embodiment, double stranded siNA molecules comprise overhanging nucleotides (e.g., 1-5 nucleotide overhangs, preferably 2 nucleotide overhangs). In a specific embodiment, the overhanging nucleotides are 3′ overhangs. In another specific embodiment, the overhanging nucleotides are 5′ overhangs. Any type of nucleotide can be a part of the overhang. In one embodiment, the overhanging nucleotide or nucleotides are ribonucleic acids. In another embodiment, the overhanging nucleotide or nucleotides are deoxyribonucleic acids. In a preferred embodiment, the overhanging nucleotide or nucleotides are thymidine nucleotides. In another embodiment, the overhanging nucleotide or nucleotides are modified or non-classical nucleotides. The overhanging nucleotide or nucleotides may have non-classical internucleotide bonds (e.g., other than phosphodiester bond).

In embodiments where the siRNA is a dsRNA, an annealing step is necessary if single-stranded RNA molecules are obtained. Briefly, combine 30.mu.1 of each RNA oligo 50.mu.M solution in 100 mM potassium acetate, 30 mM HEPES-KOH pH 7.4, 2 mM magnesium acetate. The solution is then incubated for 1 minute at 90° C. centrifuged for 15 seconds, and incubated for 1 hour at 37° C.

In embodiments where the siRNA is a short hairpin RNA (shRNA); the two strands of the siRNA molecule may be connected by a linker region (e.g., a nucleotide linker or a non-nucleotide linker).

Preparation of Polynucleotides Used for Rnai, Antisense and Ribozyme approaches. Polynucleotides for RNAi, antisense, and ribozyme approaches to reduce intracellular levels of GPR22 may be prepared by any method known in the art for the synthesis of DNA and RNA molecules. These include techniques for chemically synthesizing oligodeoxyribonucleotides and oligoribonucleotides such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. The skilled person will be aware of other types of chemical modification which may be incorporated into RNA molecules (see International Publications WO03/070744 and WO2005/045037 for an overview of types of modifications).

In one embodiment, modifications can be used to provide improved resistance to degradation or improved uptake. Examples of such modifications include phosphorothioate internucleotide linkages, 2′-O-methyl ribonucleotides (especially on the sense strand of a double stranded siRNA), 2′-deoxy-fluoro ribonucleotides, 2′-deoxy ribonucleotides, “universal base” nucleotides, 5-C-methyl nucleotides, and inverted deoxyabasic residue incorporation (see generally GB2406568).

In another embodiment, modifications can be used to enhance stability or to increase targeting efficiency. For example, with respect to siRNAs, modifications include chemical cross linking between the two complementary strands of an siRNA, chemical modification of a 3′ or 5′ terminus of a strand of an siRNA, sugar modifications, nucleobase modifications and/or backbone modifications, 2′-fluoro modified ribonucleotides and 2′-deoxy ribonucleotides (see generally International Publication WO2004/029212).

In another embodiment, for example, with respect to siRNAs, modifications can be used to increased or decreased affinity for the complementary nucleotides in the target mRNA and/or in the complementary siRNA strand (see generally International Publication WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the siNA is a double-stranded siRNA, the 3′-terminal nucleotide overhanging nucleotides are replaced by deoxyribonucleotides (see generally Elbashir et al., Genes Dev, 15:188, 2001).

Antisense polynucleotides. In further embodiments, the invention relates to the use of isolated “antisense” nucleic acids to inhibit expression, e.g., by inhibiting transcription and/or translation of a GPR22 nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides or agents facilitating transport across the cell membrane (see, e.g., Letsinger et al., Proc. Natl. Acad. Sci. USA 86:6553-6556, 1989; Lemaitre et al., Proc. Natl. Acad. Sci. USA 84:648-652, 1987; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., BioTechniques 6:958-976, 1988) or intercalating agents. (see, e.g., Zon, Pharm. Res. 5:539-549, 1988). To this end, the oligonucleotide may be conjugated to another molecule, e.g., a peptide, hybridization triggered cross-linking agent, transport agent, hybridization-triggered cleavage agent, etc.

Ribozymes. In certain embodiments, the invention relates to other nucleic acid therapies to inhibit the activity of GPR22 in colorectal cancer cells, including ribozymes, which are enzymatic RNA molecules capable of catalyzing the specific cleavage of RNA. (For a review, see Rossi, Current Biology 4:469-471, 1994) and DNA enzymes. Ribozyme molecules designed to catalytically cleave GPR22 mRNA transcripts can also be used to prevent translation of subject GPR22 mRNAs and/or expression of GPR22 polypeptides. (See, e.g., PCT International Publication WO90/11364, published Oct. 4, 1990; Sarver et al., Science 247:1222-1225, 1990; and U.S. Pat. No. 5,093,246). DNA enzymes are designed so that they recognize a particular target nucleic acid sequence, much like an antisense oligonucleotide, however much like a ribozyme they are catalytic and specifically cleave the target nucleic acid. Methods of making and administering DNA enzymes can be found, for example, in U.S. Pat. No. 6,110,462.

### Drug Discovery and Therapy

The scientific literature that has evolved around receptors has adopted a number of terms to refer to ligands having various effects on receptors. For clarity and consistency, the following definitions are used throughout this patent document. To the extent that these definitions conflict with other definitions for these terms, the following definitions shall control:

The term “agonists” as used herein refers to materials (e.g., ligands, candidate compounds) that activate an intracellular response when they bind to the receptor. In some embodiments, Agonists are those materials not previously known to activate the intracellular response when they bind to the receptor (e.g. to enhance GTPγS binding to membranes or to lower intracellular cAMP level). In some embodiments, agonists are those materials not previously known to inhibit lipolysis when they bind to the receptor.

The term “allosteric modulators” as used herein refers to materials (e.g., ligands, candidate compounds) that affect the functional activity of the receptor but which do not inhibit the endogenous ligand from binding to the receptor. Allosteric modulators include inverse agonists, partial agonists and agonists.

The term “antagonists” as used herein refers to materials (e.g., ligands, candidate compounds) that competitively bind to the receptor at the same site as the agonists but which do not activate an intracellular response, and can thereby inhibit the intracellular responses elicited by agonists. Antagonists do not diminish the baseline intracellular response in the absence of an agonist. In some embodiments, antagonists are those materials not previously known to compete with an agonist to inhibit the cellular response when they bind to the receptor, e.g. wherein the cellular response is GTPγS binding to membranes or to the lowering of intracellular cAMP level.

The term “apoptosis” (also known as Programmed Cell Death) as used herein refers to a form of cell death wherein the cell is programmed to die by signal transduction systems that operate within the cell. In contrast, necrosis is when the cell is killed by extrinsic factors.

The term “candidate compound” as used herein refers to a molecule (for example, and not limitation, a chemical compound) that is amenable to a screening technique. Preferably, the phrase “candidate compound” does not include compounds which were publicly known to be antagonists to a receptor; more preferably, not including a compound which has previously been determined to have therapeutic efficacy in at least one mammal; and, most preferably, not including a compound which has previously been determined to have therapeutic utility in humans.

The term “cardiac ejection fraction” as used herein refers to the fraction of blood ejected from the left ventricle with a single contraction. For example, if 100 ml of blood is in the left ventricle and 90 ml is ejected upon contraction, then the cardiac ejection fraction is 90%.

The term “cardiac hypertrophy” as used herein refers to enlargement of the heart muscle (myocardium). Cardiac hypertrophy is usually, but not always, an adaptive response to increased hemodynamic load imposed upon the myocardium.

The term “composition” as used herein refers to a material comprising at least one component; a “pharmaceutical composition” is an example of a composition.

The term “compound efficacy” as used herein refers to a measurement of the ability of a compound to inhibit or stimulate receptor functionality; i.e. the ability to activate/inhibit a signal transduction pathway, in contrast to receptor binding affinity. Exemplary means of detecting compound efficacy are disclosed in the Example section of this patent document.

The term “congestive heart failure” (CHF) as used herein refers to a disorder in which the heart loses its ability to pump blood efficiently. Congestive heart failure becomes more prevalent with advancing age. Ischemic heart disease is the most common cause of congestive heart failure, accounting for 60-70% of all cases. An increased venous pressure greater than 12 mmHg is one of the major Framingham criteria for congestive heart failure, as is a reduction in cardiac output equivalent to a circulation time greater than 25 seconds.

The term “constitutively active receptor” as used herein refers to a receptor stabilized in an active state by means other than through binding of the receptor to its ligand or a chemical equivalent thereof. A constitutively active receptor may be endogenous or non-endogenous. CART is an acronym for Constitutively Activated Receptor Technology and when used herein prefixing a GPCR, shall be understood to identify said prefixed GPCR as a constitutively activated receptor.

The term “constitutive receptor activation” as used herein refers to shall mean activation of a receptor in the absence of binding to its ligand or a chemical equivalent thereof.

The term “contact” as used herein refers to bringing at least two moieties together, whether in an in vitro system or an in vivo system.

The term “decrease” as used herein refers to a reduction in a measurable quantity and is used synonymously with the terms “reduce”, “decrease”, “diminish”, “lower”, and “lessen”.

The term “echocardiography” as used herein refers to a method of using sound waves to measure cardiac structure and function in living animals.

The term “endogenous” as used herein refers to a material that a mammal naturally produces. Endogenous in reference to, for example and not limitation, the term “receptor,” shall mean that which is naturally produced by a mammal (for example, and not limitation, a human). Endogenous shall be understood to encompass allelic variants of a gene represented within the genome of said mammal as well as the allelic polypeptide variants so encoded. By contrast, the term “non-endogenous” in this context shall mean that which is not naturally produced by a mammal (for example, and not limitation, a human). For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

The term “host cell” as used herein refers to a cell capable of having a vector incorporated therein. The host cell may be prokaryotic or eukaryotic. In some embodiments the host cell is eukaryotic, preferably, mammalian, and more preferably selected from the group consisting of 293, 293T, CHO and COS-7 cells. In some embodiments, the host cell is eukaryotic, more preferably melanophore.

The term “in need of treatment” as used herein refers to a judgement made by a caregiver (e.g. physician, nurse, nurse practitioner, etc. in the case of humans; veterinarian in the case of animals, including non-human mammals) that an individual or animal requires or will benefit from treatment. This judgement is made based on a variety of factors that are in the realm of a caregiver's expertise, but which include the knowledge that the individual or animal is ill, or will be ill, as the result of a condition that is treatable by the compounds of the invention.

The term “increased venous pressure” as used herein refers to the elevated blood pressure that develops in the venous system (veins) due to pooling of blood there caused by a weakening of the circulatory system.

The term “individual” as used herein refers to any animal, including mammals, preferably mice, rats, other rodents, rabbits, dogs, cats, swine, cattle, sheep, horses, or primates, and most preferably humans.

The term “inhibit,” in relationship to the term “response,” shall mean that a response is decreased or prevented in the presence of a compound as opposed to in the absence of the compound.

The term “inverse agonist” as used herein refers to materials (e.g., ligand, candidate compound) that bind either to the endogenous form or to the constitutively activated form of the receptor so as to reduce the baseline intracellular response of the receptor observed in the absence of agonists.

The term “ischemic heart disease” as used herein refers to a disorder caused by lack of oxygen to the tissues of the heart, in which muscles of the heart are affected and the heart cannot pump properly. Ischemic heart disease is the most common cardiomyopathy in the United States.

The term “isolated” as used herein shall mean that the material is removed from its original environment (e.g., the natural environment if it is naturally occurring). For example, a naturally occurring polynucleotide or polypeptide present in a living animal is not isolated, but the same polynucleotide or DNA or polypeptide, separated from some or all of the coexisting materials in the natural system, is isolated. Such a polynucleotide could be part of a vector and/or such a polynucleotide or polypeptide could be part of a composition, and still be isolated in that the vector or composition is not part of its natural environment.

The term “knockout mouse (rat, etc.)” as used herein is intended herein to encompass a mouse (or rat, etc.) that has been manipulated by recombinant means such that a single gene of choice has been inactivated or “knocked-out” in a manner that leaves all other genes unaffected.

The term “known receptor” as used herein refers to an endogenous receptor for which the endogenous ligand specific for that receptor has been identified.

The term “ligand” as used herein refers to a molecule specific for a naturally occurring receptor.

The terms “modulate” or “modify” as used herein refer to an increase or decrease in the amount, quality, or effect of a particular activity, function or molecule.

The term “myocardial infarction” as used herein refers to the damage or death of an area of heart muscle because of an inadequate supply of oxygen to that area. Myocardial infarctions are often caused by a clot that blocks one of the coronary arteries (the blood vessels that bring blood and oxygen to heart muscle). The clot prevents blood and oxygen from reaching that area of the heart, leading to the death of heart cells in that area.

The term “non-orphan receptor” as used herein refers to an endogenous naturally occurring molecule specific for an identified ligand wherein the binding of a ligand to a receptor activates an intracellular signaling pathway.

The term “orphan receptor” as used herein refers to an endogenous receptor for which the ligand specific for that receptor has not been identified or is not known.

The term “partial agonists” as used herein refers to materials (e.g., ligands, candidate compounds) that activate the intracellular response when they bind to the receptor to a lesser degree/extent than do full agonists.

The term “pharmaceutical composition” as used herein refers to a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

The term “post-myocardial infarction remodeling” is used herein in the following way. The loss of myocardial tissue due to myocardial infarction results in a sustained excessive hemodynamic burden placed on the ventricle. Ventricular hypertrophy constitutes one of the principle mechanisms by which the heart compensates for an increased load. However, the capacity for this adaptation to sustain cardiac performance in the face of hemodynamic overload is finite and, when chronically maintained, becomes maladaptive. Gradually, the adaptive hypertrophic phenotype transitions to overt heart failure as the enlarged ventricles progressively dilate and contractile function weakens. The natural history of the adaptive and maladaptive response to myocardial infarction in the heart is referred to as “remodeling.”

With regard to post-myocardial infarction remodeling, there are a number of parameters that are informative with regard to the progression of the pathology: (a) if cardiac hypertrophy increases, that is detrimental; (b) if cardiac myocyte apoptosis increases, that is detrimental; (c) if cardiac ejection fraction decreases, that is detrimental; and (d) if ventricular chamber volume increases, that is detrimental.

Measuring ejection fraction, hypertrophy, and chamber dilation can all be done in living animals with echocardiography, including in rats and mice. These parameters are typically looked at initially. In order to accurately ascertain the pathogenetic mechanisms involved, however, the animal typically further needs to be sacrificed in order to measure cardiomyocyte apoptosis.

The term “receptor functionality” as used herein refers to the normal operation of a receptor to receive a stimulus and moderate an effect in the cell, including, but not limited to regulating gene transcription, regulating the influx or efflux of ions, effecting a catalytic reaction, and/or modulating activity through G-proteins.

The term “reduced cardiac output” as used herein refers to the decreased pumping capacity of the failing heart such that less blood is pumped into the circulatory system (arteries) with each-contraction of the heart's ventricles.

The term “second messenger” as used herein refers to an intracellular response produced as a result of receptor activation. A second messenger can include, for example, inositol triphosphate (IP₃), diacylglycerol (DAG), cyclic AM (cAMP), cyclic GMP (cGMP), and Ca²⁺. Second messenger response can be measured for a determination of receptor activation. In addition, second messenger response can be measured for the direct identification of candidate compounds, including for example, inverse agonists, partial agonists, agonists, and antagonists.

The term “signal-to-noise ratio” as used herein refers to the signal generated in response to activation, amplification, or stimulation wherein the signal is above the background noise or the basal level in response to non-activation, non-amplification, or non-stimulation.

The term “spacer” as used herein refers to a translated number of amino acids that are located after the last codon or last amino acid of a gene, for example a GPCR of interest, but before the start codon or beginning regions of the G protein of interest, wherein the translated number amino acids are placed in-frame with the beginnings regions of the G protein of interest The number of translated amino acids can be one, two, three, four, etc., and up to twelve.

The term “repress” as used herein in relationship to the term “response,” shall mean that a response is decreased in the presence of a compound as opposed to in the absence of the compound.

The term “subject” as used herein refers to primates, including but not limited to humans and baboons, as well as pet animals such as dogs and cats, laboratory animals such as rats and mice, and farm animals such as horses, sheep, and cows.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “transgenic mouse (or rat, etc.)” as used herein refers to a mouse (or rat, etc.) that has been engineered through recombinant means to carry a foreign gene, or transgene, of choice as part of its own genetic material.

The term “ventricular chamber volume” as used herein refers to a measurement of the internal dimensions of the left or right ventricular chambers of the heart. In the failing heart, there is an enlargement of the ventricular chambers.

Screening of Candidate Compounds

1. Generic GPCR Screening Assay Techniques. When a G protein receptor becomes active, it binds to a G protein (e.g. Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, activated receptors continue to exchange GDP to GTP. A non-hydrolyzable analog of GTP, ³⁵S-GTPγS, can be used to monitor enhanced binding to membranes which express activated receptors. _{35S}GTPγS can be used to monitor G protein coupling to membranes in the absence and presence of ligand. An example of this monitoring, among other examples well-known and available to those in the art, was reported by Traynor and Nahorski in 1995. The preferred use of this assay system is for initial screening of candidate compounds because the system is generically applicable to all G protein-coupled receptors regardless of the particular G protein that interacts with the intracellular domain of the receptor.

2. Specific GPCR Screening Assay Techniques. Once candidate compounds are identified using the “generic” G protein-coupled receptor assay (i.e., an assay to select compounds that are antagonists), in some embodiments further screening to confirm that the compounds have interacted at the receptor site is preferred. For example, a compound identified by the “generic” assay may not bind to the receptor, but may instead merely “uncouple” the G protein from the intracellular domain.

a. Gs Gz and Gi. Gs stimulates the enzyme adenylyl cyclase. Gi (and Gz and Go), on the other hand, inhibit adenylyl cyclase. Adenylyl cyclase catalyzes the conversion of ATP to cAMP; thus, activated GPCRs that couple the Gs protein are associated with increased cellular levels of cAMP. On the other hand, activated GPCRs that couple Gi (or Gz, Go) protein are associated with decreased cellular levels of cAMP. See, generally, “Indirect Mechanisms of Synaptic Transmission,” Chap. 8, From Neuron To Brain (3rd Ed.) Nichols et al eds., Sinauer Associates, Inc., 1992. Thus, assays that detect cAMP can be utilized to determine if a candidate compound is, e.g., an anagonist to the receptor. A variety of approaches known in the art for measuring cAMP can be utilized; in some embodiments a preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, an activated Gs-linked receptor causes the accumulation of cAMP that then activates the gene and expression of the reporter protein. The reporter protein such as β.-galactosidase or luciferase can then be detected using standard biochemical assays.

b. Go and Gq. Gq and Go are associated with activation of the enzyme phospholipase C, which in turn hydrolyzes the phospholipid PIP₂, releasing two intracellular messengers: diacyclglycerol (DAG) and inistol 1,4,5-triphoisphate (IP₃). Increased accumulation of IP₃ is associated with activation of Gq- and Go-associated receptors. See, generally, “Indirect Mechanisms of Synaptic Transmission,” Chap. 8, From Neuron To Brain (3rd Ed.) Nichols et al eds. Sinauer Associates, Inc., 1992. Assays that detect IP₃ accumulation can be utilized to determine if a candidate compound is, e.g., an antagonist to a Gq- or Go-associated receptor. Gq-associated receptors can also been examined using an AP1 reporter assay in that Gq-dependent phospholipase C causes activation of genes containing AP1 elements; thus, activated Gq-associated receptors will evidence an increase in the expression of such genes, whereby antagonists and inverse agonists thereto will evidence a decrease in such expression, and agonists will evidence an increase in such expression. Commercially available assays for such detection are available.

4. Co-Transfection of a Target Gi Coupled GPCR with a Signal-Enhancer Gs Coupled GPCR (cAMP Based Assays). A Gi coupled receptor is known to inhibit adenylyl cyclase, and, therefore, decreases the level of cAMP production, which can make the assessment of cAMP levels challenging. In some embodiments, an effective technique in measuring the decrease in production of cAMP as an indication of activation of a receptor that predominantly couples Gi upon activation can be accomplished by co-transfecting a signal enhancer, e.g., a non-endogenous, constitutively activated receptor that predominantly couples with Gs upon activation (e.g., TSHR-A6231; see infra), with the Gi linked GPCR. As is apparent, activation of a Gs coupled receptor can be determined based upon an increase in production of cAMP. Activation of a Gi coupled receptor leads to a decrease in production cAMP. Thus, the co-transfection approach is intended to advantageously exploit these “opposite” affects. For example, co-transfection of a non-endogenous, constitutively activated Gs coupled receptor (the “signal enhancer”) with expression vector alone provides a baseline cAMP signal (i.e., although the Gi coupled receptor will decrease cAMP levels, this “decrease” will be relative to the substantial increase in cAMP levels established by constitutively activated Gs coupled signal enhancer). By then co-transfecting the signal enhancer with the “target receptor”, an antagonist or inverse agonist of the Gi coupled target receptor will increase the measured cAMP signal, while an agonist of the Gi coupled target receptor will decrease this signal.

Candidate compounds that are directly identified using this approach should be assessed independently to ensure that these do not target the signal enhancing receptor (this can be done prior to or after screening against the co-transfected receptors).

### C. Medicinal Chemistry

Candidate Compounds. Any molecule known in the art can be tested for its ability to modulate (increase or decrease) the activity of a GPCR of the present invention. For identifying a compound that modulates activity, candidate compounds can be directly provided to a cell expressing the receptor.

This embodiment of the invention is well suited to screen chemical libraries for molecules which modulate, e.g., inhibit, antagonize, or agonize, the amount of, or activity of, a receptor. The chemical libraries can be peptide libraries, peptidomimetic libraries, chemically synthesized libraries, recombinant, e.g., phage display libraries, and in vitro translation-based libraries, other non-peptide synthetic organic libraries, etc. This embodiment of the invention is also well suited to screen endogenous candidate compounds comprising biological materials, including but not limited to plasma and tissue extracts, and to screen libraries of endogenous compounds known to have biological activity.

In some embodiments direct identification of candidate compounds is conducted in conjunction with compounds generated via combinatorial chemistry techniques, whereby thousands of compounds are randomly prepared for such analysis. The candidate compound may be a member of a chemical library. This may comprise any convenient number of individual members, for example tens to hundreds to thousands to millions of suitable compounds, for example peptides, peptoids and other oligomeric compounds (cyclic or linear), and template-based smaller molecules, for example benzodiazepines, hydantoins, biaryls, carbocyclic and polycyclic compounds (e.g., naphthalenes, phenothiazines, acridines, steroids etc.), carbohydrate and amino acid derivatives, dihydropyridines, benzhydryls and heterocycles (e.g., trizines, indoles, thiazolidines etc.). The numbers quoted and the types of compounds listed are illustrative, but not limiting. Preferred chemical libraries comprise chemical compounds of low molecular weight and potential therapeutic agents.

Exemplary chemical libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Further, more general, structurally constrained, organic diversity (e.g., nonpeptide) libraries, can also be used. By way of example, a benzodiazepine library (see e.g., Bunin et al., Proc. Natl. Acad. Sci. USA 91:4708-4712, 1994) may be used.

In another embodiment of the present invention, combinatorial chemistry can be used to identify modulators of the GPCRs of the present invention. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See, e.g., Matter, J. Med. Chem. 40:1219-1229, 1997).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, the receptors of the present invention). The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., Chemistry and Biology 2:107-118, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Candidate Compounds Identified as Modulators. Generally, the results of such screening will be compounds having unique core structures; thereafter, these compounds may be subjected to additional chemical modification around a preferred core structure(s) to further enhance the medicinal properties thereof. Such techniques are known to those in the art and will not be addressed in detail in this patent document.

In some embodiments, said identified modulator is bioavailable. A number of computational approaches available to those of ordinary skill in the art have been developed for prediction of oral bioavailability of a drug [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is hereby incorporated by reference in its entirety). Furthermore, positron emission tomography (PET) has been successfully used by a number of groups to obtain direct measurements of drug distribution, including an assessment of oral bioavailability, in the mammalian body following oral administration of the drug, including non-human primate and human body (Noda et al., J. Nucl. Med. (2003) 44:105-108, 2002; Gulyas et al., Eur. J. Nucl. Med. Mol. Imaging. 29:1031-8; Kanerva et al., Psychopharmacol. 145:76-81, 1999).

### Neuroprotective Compositions and Methods

Due to the enriched expression of GPR22 to the adult brain and heart, repression of GPR22 expression or activity, is expected to be benefitial for neural or behavioral disorders. Reduction of GPR22 activity or expression, will be also beneficial as a protective strategy to neuronal degenerative disease.

As used herein, “neurological disorder” refers to any disorder of the nervous system and/or visual system. “Neurological disorders” include disorders that involve the central nervous system (brain, brainstem and cerebellum), the peripheral nervous system (including cranial nerves), and the autonomic nervous system (parts of which are located in both central and peripheral nervous system). Major groups of neurological disorders include, but are not limited to, headache, stupor and coma, dementia, seizure, sleep disorders, trauma, infections, neoplasms, neuroopthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological disorder. The following is a list of several neurological disorders, symptoms, signs and syndromes that can be treated using compositions and methods according to the present invention: acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; and Zellweger syndrome.

The neurological disorder can be an affective disorder (e.g., depression or anxiety). As used herein, “affective disorder” or “mood disorder” refers to a variety of conditions characterized by a disturbance in mood as the main feature. If mild and occasional, the feelings may be normal. If more severe, they may be a sign of a major depressive disorder or dysthymic reaction or be symptomatic of bipolar disorder. Other mood disorders may be caused by a general medical condition. See, e.g., Mosby's Medical, Nursing & Allied Health Dictionary, 5^{th} edition (1998).

As used herein, “depression” refers to an abnormal mood disturbance characterized by feelings of sadness, despair, and discouragement. Depression refers to an abnormal emotional state characterized by exaggerated feelings of sadness, melancholy, dejection, worthlessness, emptiness, and hopelessness, that are inappropriate and out of proportion to reality. See, Mosby's Medical, Nursing & Allied Health Dictionary, 5^{th} edition (1998). Depression includes, but is not limited to: a major depressive disorder (single episode, recurrent, mild, severe without psychotic features, severe with psychotic features, chronic, with catatonic features, with melancholic features, with atypical features, with postpartum onset, in partial remission, in full remission), dysthymic disorder, adjustment disorder with depressed mood, adjustment disorder with mixed anxiety and depressed mood, premenstrual dysphoric disorder, minor depressive disorder, recurrent brief depressive disorder, post-psychotic depressive disorder of schizophrenia, a major depressive disorder associated with Parkinson's disease, and a major depressive disorder associated with dementia.

The neurological disorder can be pain-associated depression (PAD). As used herein, “pain-associated depression” or “PAD” is intended to refer to a depressive disorder characterized by the co-morbidity of pain and atypical depression. Specifically, the pain can be chronic pain, neuropathic pain, or a combination thereof. Specifically, the PAD can include atypical depression and chronic pain wherein the chronic pain precedes the atypical depression, or vice versa.

“Chronic pain” refers to pain that continues or recurs over a prolonged period of time (i.e., greater than three months), caused by various diseases or abnormal conditions, such a rheumatoid arthritis, for example. Chronic pain may be less intense than acute pain. A person with chronic pain does not usually display increased pulse and rapid perspiration because the automatic reactions to pain cannot be sustained for long periods of time. Others with chronic pain may withdraw from the environment and concentrate solely on their afflicton, totally ignoring their family and friends and external stimuli. See, e.g., Mosby's Medical, Nursing & Allied Health Dictionary, 5^{th} edition (1998).

Chronic pain includes but is not limited to: lower back pain, atypical chest pain, headache, pelvic pain, myofascial face pain, abdominal pain, and neck pain or chronic pain caused by disease or a condition such as, for example, arthritis, temporal mandibular joint dysfunction syndrome, traumatic spinal cord injury, multiple sclerosis, irritable bowel syndrome, chronic fatigue syndrome, premenstrual syndrome, multiple chemical sensitivity, closed head injury, fibromyalgia, rheumatoid arthritis, diabetes, cancer, HIV, interstitial cystitis, migraine headache, tension headache, post-herpetic neuralgia, peripheral nerve injury, causalgia, post-stroke syndrome, phantom limb syndrome e, and chronic pelvic pain.

“Atypical depression” refers to a depressed affect, with the ability to feel better temporarily in response to positive life effect (mood reactivity), plus two or more neurovegetative symptoms, including, but not limited to: hypersomnia, increased appetite or weight gain, leaden paralysis, and a long-standing pattern of extreme sensitivity to perceived interpersonal rejection; wherein the neurovegetative symptoms are present for more than about two weeks. Such neurovegetative symptoms can be reversed compared to those found in other depressive disorders (e.g., melancholic depression).

“Acute neurological disorder” refers to a neurological disorder having a rapid onset followed by a short but severe course, including, but not limited to, febrile seizures, Guillain-Barré syndrome, stroke, and intracerebral hemorrhaging.

“Chronic neurological disorder” refers to a neurological disorder lasting for a long period of time (e.g., more than about two weeks; specifically, the chronic neurological disorder can continue or recur for more than about four weeks, more than about eight weeks, or more than about twelve weeks) or is marked by frequent recurrence, including, but not limited to, narcolepsy, chronic inflammatory demyelinating polyneuropathy, cerebral palsy, epilepsy, multiple sclerosis, dyslexia, Alzheimer's disease, and Parkinson's disease.

“Trauma” refers to any injury or shock to the body, as from violence or an accident, or to any emotional wound or shock, such as a wound or shock that causes substantial, lasting damage to the psychological development of a person.

“Ischemic condition” is any condition that results in a decrease in the blood supply to a bodily organ, tissue or part caused by constriction or obstruction of the blood vessels, often resulting in a reduction of oxygen to the organ, tissue or part.

“Hypoxic conditions” are conditions in which the amount or concentration of oxygen in the air, blood or tissue is low (subnormal).

“Painful neuropathy” or “neuropathy” is chronic pain that results from damage to or pathological changes of the peripheral or central nervous system. Peripheral neuropathic pain is also referred to as painful neuropathy, nerve pain, sensory peripheral neuropathy, or peripheral neuritis. With neuropathy, the pain is not a symptom of injury but rather is itself the disease process. Neuropathy is not associated with the healing process. Rather than communicating that there is an injury somewhere, the nerves themselves malfunction and become the cause of pain.

“Neuropathic pain” refers to pain associated with inflammation or degeneration of the peripheral nerves, cranial nerves, spinal nerves, or a combination thereof. The pain is typicalloy sharp, stinging, or stabbing. The underlying disorder can result in the destruction of peripheral nerve tissue and can be accompanied by changes in skin color, temperature and edema. See, e.g., Mosby's Medical, Nursing & Allied Health Dictionary, 5^{th} edition (1998); and Stedman's Medical Dictionary, 25^{th} edition (1990).

“Diabetic neuropathy” refers to a peripheral nerve disorder/nerve damage caused by diabetes, including peripheral, autonomic, and cranial nerve disorders/damage associated with diabetes. Diabetic neuropathy is a common complication of diabetes mellitus in which nerves are damaged as a result of hyperglycemia (high blood sugar levels).

“Drug dependence” refers to habituation to, abuse of, and/or addiction to a chemical substance. Largely because of psychological craving, the life of the drug-dependent person revolves around the need for the specific effect of one or more chemical agents on mood or state of consciousness. The term thus includes not only the addiction (which emphasizes the physiological dependence) but also drug abuse (in which the pathological craving for drugs seem unrelated to physical dependence). Examples include, but are not limited to, dependence on alcohol, opiates, synthetic analgesics with morphine-like effects, barbiturates, hypnotics, sedatives, some antianxiety agents, cocaine, psychostimulants, marijuana, nicotine and psychotomimetic drugs.

“Drug withdrawal” refers to the termination of drug taking. Drug withdrawal also refers to the clinical syndrome of psychological and, sometimes, physical factors that result from the sustained use of a particular drug when the drug is abruptly withdrawn. Symptoms are variable but may include anxiety, nervousness, irritability, sweating, nausea, vomiting, rapid heart rate, rapid breathing, and seizures.

“Drug addiction” or dependence is defined as having one or more of the following signs: a tolerance for the drug (needing increased amounts to achieve the same effect), withdrawal symptoms, taking the drug in larger amounts than was intended or over a longer period of time than was intended, having a persistent desire to decrease or the inability to decrease the amount of the drug consumed, spending a great deal of time attempting to acquire the drug, or continuing to use the drug even though the person knows there are recurring physical or psychological problems caused by the drug.

“Depression” refers to a mental state of depressed mood characterized by feelings of sadness, despair and discouragement. Depression ranges from normal feelings of the blues through dysthymia to major depression.

“Anxiety disorders” refers to an excessive or inappropriate aroused state characterized by feelings of apprehension, uncertainty, or fear. Anxiety disorders have been classified according to the severity and duration of their symptoms and specific behavioral characteristics. Categories include: generalized anxiety disorder, which is long-lasting and low-grade; panic disorder, which has more dramatic symptoms; phobias; obsessive-compulsive disorder; post-traumatic stress disorder; and separation anxiety disorder.

“Tardive dyskinesia” (e.g., Tourette's syndrome) refers to a serious, irreversible neurological disorder that can appear at any age. Tardive dyskinesia can be a side effect of long-term use of antipsychotic/neuroleptic drugs. Symptoms can be hardly noticeable or profound. Symptoms involve uncontrollable movement of various body parts, including the body, trunk, legs, arms, fingers, mouth, lips, or tongue.

“Movement disorder” refers to a group of neurological disorders that involve the motor and movement systems, including, but not lomited to, ataxia, Parkinson's disease, blepharospasm, Angelman syndrome, ataxia telangiectasia, dysphonia, dystonic disorders, gait disorders, torticollis, writer's cramp, progressive supranuclear palsy, Huntington's chorea, Wilson's disease, myoclonus, spasticitiy, tardive dyskinesia, tics, Tourette syndrome, and tremors.

“Cerebral infections that disrupt the blood-brain barrier” refers to infections of the brain or cerebrum that result in an alteration in the effectiveness of the blood-brain barrier, either increasing or decreasing its ability to prevent substances and/or organisms from passing out of the bloodstream and into the central nervous system.

“Blood-brain barrier” refers to a semi-permeable layer of endothelial cells within capillaries of the central nervous system that prevents large molecules, immune cells, many potentially damaging substances, and foreign organisms (e.g., viruses) from passing out of the bloodstream and into the central nervous system (e.g., brain and spinal cord). A dysfunction in the blood-brain barrier may underlie in part the disease process in multiple sclerosis.

“Meningitis” refers to inflammation of the meninges of the brain and spinal cord, most often caused by a bacterial or viral infection and characterized by fever, vomiting, intense headache, and stiff neck.

“Meningoencephalitis” refers to inflammation of one or both of the brain and meninges.

“Stroke,” also called cerebral accident or cerebrovascular accident, refers to a sudden loss of brain function caused by a blockage or rupture of a blood vessel to the brain (resulting in a lack of oxygen to the brain), characterized by loss of muscular control, diminution or loss of sensation or consciousness, dizziness, slurred speech, or other symptoms that vary with the extent and severity of the damage to the brain.

“Hypoglycemia” refers to an abnormally low level of glucose in the blood.

“Cerebral ischemia” (stroke) refers to a deficiency in blood supply to the brain, often resulting in a lack of oxygen to the brain.

“Cardiac arrest” refers to a sudden cessation of heartbeat and cardiac function, resulting in a temporary or permanent loss of effective circulation.

“Spinal cord trauma,” also called spinal cord injury or compression, refers to damage to the spinal cord that results from direct injury to the spinal cord itself or indirectly by damage to the bones and soft tissues and vessels surrounding the spinal cord.

“Head trauma” refers to a head injury of the scalp, skull, or brain. These injuries can range from a minor bump on the skull to a devastating brain injury. Head trauma can be classified as either closed or penetrating. In a closed head injury, the head sustains a blunt force by striking against an object. A concussion is a closed head injury that involves the brain. In a penetrating head injury, an object (usually moving at high speed, such as a windshield or other part of a motor vehicle) breaks through the skull and enters the brain.

“Perinatal hybpxia” refers to a lack of oxygen during the perinatal period (i.e., the period of time occurring shortly before and after birth, variously defined as beginning with completion of the twentieth to twenty-eighth week of gestation and ending 7 to 28 days after birth.

“Hypoglycemic neuronal damage” refers to neuronal damage, for example, nerve damage, resulting from a hypoglycemic condition (i.e., abnormally low blood glucose levels).

“Neurodegenerative disorder” refers to a type of neurological disease marked by the loss of nerve cells, including, but not limited to, Alzheimer's disease, Parkinson's disease, amyotrophic lateral sclerosis, tauopathies (including fronto-temporal dementia), and Huntington's disease.

“Epilepsy” refers to any of various neurological disorders characterized by sudden recurring attacks of motor, sensory, or psychic malfunction with or without loss of consciousness or convulsive seizures.

“Alzheimer's disease” refers to a disease marked by the loss of cognitive ability, generally over a period of 10 to 15 years, and associated with the development of abnormal tissues and protein deposits (plaques or tangles) in the cerebral cortex.

“Huntington's disease” refers to a hereditary disease that develops in adulthood and ends in dementia. It results from genetically programmed neuronal degeneration in certain areas of the brain that causes uncontrolled movements, loss of intellectual faculties, and emotional disturbance.

“Parkinsonism” refers to a disorder similar to Parkinson's disease, but which is caused by the effects of a medication, a different neurodegenerative disorder, or another illness. The term “parkinsonism” also refers to any condition that causes any combination of the types of movement abnormalities seen in Parkinson's disease by damaging or destroying dopamine neurons in a certain area of the brain.

“Amyotrophic lateral sclerosis” (ALS), also called Lou Gehrig's disease, refers to a progressive, fatal neurological disease. ALS belongs to a class of disorders known as motor neuron disease. ALS occurs when specific nerve cells in the brain and spinal cord that control voluntary movement gradually degenerate (usually the “upper” (i.e., in the cerebrocortex) and “lower” (in the spinal cord) motor neurons. The loss of these motor neurons causes the muscles under their control to weaken and waste away, leading to paralysis. ALS manifests itself in different ways, depending on which muscles weaken first. Symptoms may include tripping and falling, loss of motor control in hands and arms, difficulty speaking, swallowing and/or breathing, persistent fatigue, and twitching and cramping, sometimes quite severely. Upper motor neuron variants (e.g., primary lateral sclerosis) are also included.

“Glaucoma” refers to any of a group of eye diseases characterized by abnormally high intraocular fluid pressure, damaged optic disk, hardening of the eyeball, and partial to complete loss of vision. The retinal ganglion cells are lost in glaucoma. Some variants of glaucoma (low tension glaucoma) have normal intraocular pressure.

“Retinal ischemia” refers to a decrease in the blood supply to the retina.

“Ischemic optic neuropathy” refers to a condition that usually presents with a sudden onset of unilaterally reduced vison. The condition is the result of decreased blood flow to the optic nerve (ischemia). There are two basic types: arteritic and non-arteritic ischemic optic neuropathy. Non-arteritic ischemic optic neuropathy is generally the result of cardiovascular disease. Patients at greatest risk have a history of high blood pressure, elevated cholesterol, smoking, diabetes, or combinations of these. Arteritic ischemic optic neuropathy is caused by the inflammation of vessels supplying blood to the optic nerves, known as temporal arteritis. This condition usually presents with sudden and severe vision loss in one eye, pain in the jaw with chewing, tenderness in the temple area, loss of appetite, and a generalized felling of fatigue or illness.

“Macular degeneration” refers to the physical disturbance of the center of the retina called the macula, leading to a loss of central vision, although color vision and peripheral vision may remain clear. Vision loss usually occurs gradually and typically affects both eyes at different rates.

A “demyelinating disorder” is a condition resulting from damage to the myelin sheath, which surrounds nerves and is responsible for efficient transmission of nerve impulses to the brain. A demyelinating disorder may result in muscle weakness, poor coordination and possible paralysis. Examples of demyelinating disorders include, but are not limited to: multiple sclerosis, optic neuritis, transverse neuritis and Guillain-Barré syndrome. When treating a demyelinating disorder, a composition according to the present invention may include an N-methyl-D-aspartate-type glutamate receptor (NMDAR) antagonist (e.g., memantine) or beta interferon isoforms, copaxone or Antegren (natalizumab). Since neuronal damage may occur in demyelanting conditions such as multiple sclerosis, useful drug compositiosn may also protect the neuron instead of or in addition to the myelin.

“Multiple sclerosis” refers to a chronic disease of the central nervous system, which predominantly affects young adults and is characterized by areas of demyelination and T-cell predominant perivascular inflammation in the white matter of the brain. Some axons may be spared from these pathological processes. The disease begins most commonly with acute or subacute onset of neurologic abnormalities. Initial and subsequent symptoms may dramatically vary in their expression and severity over the course of the disease, which usually lasts for many years. Early symptoms may include numbness and/or paresthesia, mono- or paraparesis, double vision, optic neuritis, ataxia and bladder control problems. Subsequent symptoms also include more prominent upper motor neuron signs, i.e., increased spasticity, increasing para- or quadriparesis. Vertigo, incoordination and other cerebellar problems, depression, emotional lability, abnormalities in gait, dysarthria, fatigue and pain are also commonly seen.

“Sequelae of hyperhomocystinemia” refers to a condition following as a consequence hyperhomocystinemia, i.e., elevated levels of homocysteine.

“Convulsion” refers to a violent involuntary contraction or series of contractions of the muscles.

“Pain” refers to an unpleasant sensation associated with actual or potential tissue damage that is mediated by specific nerve fibers to the brain where its conscious appreciation may be modified by various factors. See, e.g., Mosby's Medical, Nursing & Allied Health Dictionary, 5^{th} edition, 1998; and Stedman's Medical Dictionary, 25^{th} edition, 1990.

“Anxiety” refers to a state of apprehension, uncertainty, and/or fear resulting from the anticipation of a realistic or fantasized threatening event or situation, often impairing physical and psychological functioning.

“Schizophrenia” refers to any of a group of psychotic disorders usually characterized by withdrawal from reality, illogical patterns of thinking, delusions, and hallucinations, and accompanied in varying degrees by other emotional, behavioral, or intellectual disturbances. Schizophrenia is associated with dopamine imbalances in the brain and defects of the frontal lobe.

“Muscle spasm” refers to an often painful involuntary muscular contraction.

“Migraine headache” refers to a severe, debilitating headache often associated with photophobia and blurred vision.

“Urinary incontinence” refers to the inability to control the flow of urine and involuntary urination.

“Nicotine withdrawal” refers to the withdrawal from nicotine, an addictive compound found in tobacco, which is characterized by symptoms that include headache, anxiety, nausea and a craving for more tobacco. Nicotine creates a chemical dependency, so that the body develops a need for a certain level of nicotine at all times. Unless that level is maintained, the body will begin to go through withdrawal.

“Opiate tolerance” refers to a homeostatic response that reduces the sensitivity of the system to compensate for continued exposure to high levels of an opiate, e.g., heroine or morphine. When the drug is stopped, the system is no longer as sensitive to the soothing effects of the enkephalin neurons and the pain of withdrawal is produced.

“Opiate withdrawal” refers to an acute state caused by cessation or dramatic reduction of use of opiate drugs that has been heavy and prolonged (several weeks or longer). Opiates include heroin, morphine, codeine, Oxycontin, Dilaudid, methadone and others. Opiate withdrawal often includes sweating, shaking, headache, drug craving, nausea, vomiting, abdominal cramping, diarrhea, inability to sleep, confusion, agitaton, depression, anxiety, and other behavioral changes.

“Emesis” refers to the act of vomiting.

“Brain edema” refers to an excessive accumulation of fluid in, on, around and/or in relation to the brain.

“AIDS— (or HIV-) induced (or associated) dementia” refers to dementia (a deterioration of intellectual faculties, such as memory, concentration, and judgment, resulting from an organic disease or disorder of the brain) induced by human immunodeficiency virus (HIV), which causes acquired immunodeficiency syndrome (AIDS).

“HIV-related neuropathy” refers to a neuropathy in a mammal infected with HIV where the neuropathy is caused by infections such as with CMV or other viruses of the herpes family. Neuropathy is the name given to a group of disorders whose symptoms may range from a tingling sensation or numbness in the toes and fingers to pain to paralysis.

“Ocular damage” refers to any damage to the eyes or in relation to the eyes.

“Retinopathy” refers to any pathological disorder of the retina.

“Cognitive disorder” refers to any cognitive dysfunction, for example, disturbance of memory (e.g., amnesia) or learning.

### Pharmaceutical Compositions

The invention provides methods of treatment (and prevention) by administration to an individual in need of said treatment (or prevention) a therapeutically effect amount of an substance that reduces GPR22 activity in a cell, tissue or organism (i.e., a “modulator”) (also see, e.g., PCT Application Number WO 02/066505). In a preferred aspect, the substance is purified. The individual is preferably an animal including, but not limited to animals such as cows, pigs, horses, chickens, cats, dogs, rabbits, rats, mice, etc., and is preferably a mammal, and most preferably human.

Modulators of the invention can be administered to humans or non-human animals, alone or in pharmaceutical or physiologically acceptable compositions where they are mixed with suitable carriers or excipient(s) using techniques well known to those in the art. Suitable pharmaceutically-acceptable carriers are available to those in the art; for example, see Remington's Pharmaceutical Sciences, 16.sup.th Edition, 1980, Mack Publishing Co., (Oslo et al., eds.).

The pharmaceutical or physiologically acceptable composition is then provided at therapeutically effect dose. A therapeutically effective dose refers to that amount of a modulator sufficient to result in prevention or amelioration of symptoms or physiological status of a cardiovascular condition, disease or injury, including but not limited to an ischemic heart disease, including myocardial infarction, post-myocardial infarction remodeling, and congestive heart failure. In some embodiments, a therapeutically effective dose refers to that amount of a modulator sufficient to result in prevention or amelioration of symptoms or physiological status of a cardiovascular disorder, including reduced cardiac output and increased venous pressures as determined illustratively and not by limitation by the methods described herein. In some embodiments, a therapeutically effective dose refers to that amount of a modulator sufficient to effect a needed change in cardiovascular function, including a decrease in cardiac hypertrophy, an increase in cardiac ejection volume, a decrease in ventricular chamber volume, and a decrease in cardiomyocyte apoptosis as determined illustratively and not by limitation by the methods described herein.

The modulators of the invention may be provided alone or in combination with other pharmaceutically or physiologically acceptable compounds. Other compounds for the treatment of disorders of the invention are currently well known in the art. One aspect of the invention encompasses the use according to embodiments disclosed herein further comprising one or more agents selected from the group consisting of captopril, enalapril maleate, lininopril, ramipril, perindopril, furosemide, torasemide, chlorothiazide, hydrochlorothiazide, amiloride hydrochloride, spironolactone, atenolol, bisoprolol, carvedilol, metoprolol tartrate, and digoxin.

In some embodiments the ischemic heart disease is selected from the group consisting of myocardial infarction, post-myocardial infarction remodeling, and congestive heart failure. In some embodiments, the cardiovascular disorder is selected from the group consisting of reduced cardiac output and increased venous pressures. In some embodiments, the needed change in cardiovascular function is selected from the group consisting of a decrease in cardiac hypertrophy, an increase in cardiac ejection volume, a decrease in ventricular chamber volume, and a decrease in cardiomyocyte apoptosis.

Routes of Administration. Suitable routes of administration include oral, nasal, rectal, transmucosal, or intestinal administration, parenteral delivery, including intramuscular, subcutaneous, intramedullary injections, as well as intrathecal, direct intraventricular, intravenous, intraperitoneal, intranasal, intrapulmonary (inhaled) or intraocular injections using methods known in the art. Other particularly preferred routes of administration are aerosol and depot formulation. Sustained release formulations, particularly depot, of the invented medicaments are expressly contemplated. In some embodiments, route of administration is oral.

Composition/Formulation. Pharmaceutical or physiologically acceptable compositions and medicaments for use in accordance with the present invention may be formulated in a conventional manner using one or more physiologically acceptable carriers comprising excipients and auxiliaries. Proper formulation is dependent upon the route of administration chosen.

Certain of the medicaments described herein will include a pharmaceutically or physiologically acceptable carrier and at least one modulator of the invention. For injection, the agents of the invention may be formulated in aqueous solutions, preferably in physiologically compatible buffers such as Hanks's solution, Ringer's solution, or physiological saline buffer such as a phosphate or bicarbonate buffer. For transmucosal administration, penetrants appropriate to the barrier to be permeated are used in the formulation. Such penetrants are generally known in the art.

Pharmaceutical or physiologically acceptable preparations that can be taken orally include push-fit capsules made of gelatin, as well as soft, sealed captulse made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs for a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for ue in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage for, e.g., in ampoules or in multi-dose containers, with an added preservative. The compositions may take such forms as suspension, solutions or emulsions in aqueous vehicles, and may contain formulatory agents such as suspending, stabilizing and/or dispersing agents.

Pharmaceutical or physiologically acceptable formulations for parenteral administration include aqueous solutions of the active compounds in water-soluble form. Aqueous suspension may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In a particular embodiment, the compounds can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, N.Y., 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). Other controlled release systems are discussed in the review by Langer (1990, Science 249:1527-1533).

Additionally, the compounds may be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for modulator stabilization may be employed.

The pharmaceutical or physiologically acceptable compositions also may comprise suitable solid or gel phase carriers or excipients. Examples of such carriers or escipients include but are not limited to calcium carbonate, calcium phosphate, various sugars, starches, cellulos derivatives, gelatin, and polymers such as polyethylene glycols.

Effective Dosage. Pharmaceutical or physiologically acceptable compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated, as may be determined using well-known methods by the skilled person.

For any compound used in the method of the invention, the therapeutically effective dose can be estimated initially from cell culture assays. For example, a dose can be formulated in animal models to achieve a circulating concentration range that includes or encompasses a concentration point or range shown to cell death-protective in an in vitro system. [See Examples, infra, for in vitro assays and in vivo animal models.] Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD₅₀ (the dose lethal to 50% of the test population) and the ED.sub.50 (the dose therapeutically effective in 50% of the test population). The dose ratio between toxic and therapeutic effects is the therapeutic index and it can be expressed as the ratio between LD₅₀ and ED₅₀. Compounds that exhibit high therapeutic indices are preferred.

The data obtained from these cell culture assays and animal studies can be used in formulating a range of dosage for use in humans. The dosage of such compounds lies preferably within a range of circulating concentrations that include the ED.sub.50, with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1).

Dosage amount and interval may be adjusted individually to provide plasma levels of the active compound which are sufficient to prevent or treat a disorder of the invention, depending on the particular situation. Dosages necessary to achieve these effects will depend on individual characteristics and route of administration.

Dosage intervals can also be determined using the value for the minimum effective concentration. Compounds should be administered using a regimen that maintains plasma levels above the minimum effective concentration for 10-90% of the time, preferably between 30-99%, and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration, and the judgement of the prescribing physician.

A preferred dosage range for the amount of a modulator of the invention, which can be administered on a daily or regular basis to achieve desired results, including but not limited to prevention or treatment of an ischemic heart disease of the invention, prevention or treatment of a cardiovascular disorder of the invention, or the effecting of a needed change in cardiovascular function of the invention, is 0.1-100 mg/kg body mass. Other preferred dosage range is 0.1-30 mg/kg body mass. Other preferred dosage range is 0.1-10 mg/kg body mass. Other preferred dosage range is 0.1-3.0 mg/kg body mass. Of course, these daily dosages can be delivered or administered in small amounts periodically during the course of a day. It is noted that these dosage ranges are only preferred ranges and are not meant to be limiting to the invention.

### E. Methods of Treatment

The invention is drawn inter alia to methods of preventing or treating a cardiovascular condition, disease or injury, including but not limited to an ischemic heart disease, including myocardial infarction, post-myocardial infarction remodeling, and congestive heart failure, comprising providing an individual in need of such treatment with a modulator of the invention. The invention is also drawn inter alia to methods of preventing or treating a cardiovascular disorder, including reduced cardiac output and increased venous pressures, comprising providing an individual in need of such treatment with a modulator of the invention. The invention is also drawn inter alia to methods of effecting a needed change in cardiovascular function, including a decrease in cardiac hypertrophy, an increase in cardiac ejection volume, a decrease in ventricular chamber volume, and a decrease in cardiomyocyte apoptosis, comprising providing an individual in need of such treatment with a modulator of the invention. In some embodiments, said modulator is orally bioavailable. In some embodiments, the modulator is provided to the individual in a pharmaceutical composition that is taken orally. Preferably the individual is a mammal, and most preferably a human.

### F. Other Utility

Agents that modulate (i.e., increase, decrease, or block) cardiomyocyte-protective GPR22 receptor functionality may be identified by contacting a candidate compound with GPR22 receptor and determining the effect of the candidate compound on GPR22 receptor functionality. The selectivity of a compound that modulates the functionality of GPR22 receptor can be evaluated by comparing its effects on GPR22 receptor to its effects on other receptors. Following identification of compounds that modulate GPR22 receptor functionality, such candidate compounds may be further tested in other assays including, but not limited to, in vivo models, in order to confirm or quantitate their activity. Modulators of GPR22 receptor functionality will be therapeutically useful in treatment of diseases and physiological conditions in which normal or aberrant GPR22 receptor functionality is involved.

Agents that are modulators (i.e., increase, decrease, or block) of cardioprotection may be identified by contacting a candidate compound with a GPR22 receptor and determining the effect of the candidate compound on GPR22 receptor functionality. In some embodiments, said cardioprotection comprises prevention or reduction of cardiomyocyte death. In some embodiments, said cardiomyocyte death comprises cardiomyocyte apoptosis. In some embodiments, said cardioprotection comprises myocardial protection against ischemia. In some embodiments, said cardioprotection comprises reduced size of infarction. In some embodiments, said cardioprotection comprises improved postischemic contractile recovery. In some embodiments, said cardioprotection comprises suppression of malignant ischemia-induced arrhythmias. The selectivity of a compound that modulates the functionality of GPR22 receptor can be evaluated by comparing its effects on GPR22 receptor to its effects on other receptors. Following identification of compounds that modulate GPR22 receptor functionality, such candidate compounds may be further tested in other assays including, but not limited to, in vivo models, in order to confirm or quantitate their activity. Modulators of GPR22 receptor functionality will be therapeutically useful in treatment of diseases and physiological conditions in which normal or aberrant GPR22 functionality is involved.

The present invention also relates to radioisotope-labeled versions of compounds of the invention identified as modulators or ligands of GPR22 that would be useful not only in radio-imaging (see, e.g., Lemstra et al., Gerontology 49:55-60, 2003; Myers et al., J. Psychopharmacol. 13:352-357, 1999), but also in assays, both in vitro and in vivo, for localizing and quantitating GPR22 in tissue samples, including human, and for identifying GPR22 ligands by inhibition binding of a radioisotope-labeled compound. It is a further object of this invention to develop novel GPR22 assays of which comprise such radioisotope-labeled compounds. By way of illustration and not limitation, it is envisioned that visualization of GPR22 through radio-imaging may identify an individual at risk for or progressing toward ischemic heart disease, including myocardial infarction, post-myocardial infarction remodeling, and congestive heart failure.

The present invention embraces radioisotope-labeled versions of compounds of the invention identified as modulators or ligands of GPR22.

In some embodiments, a radioisotope-labeled version of a compound is identical to the compound, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (deuterium), ³H (tritium), ¹¹C, ¹³C, ¹⁴C, ¹³N, ¹⁵N, ¹⁵O, ¹⁷O, ¹⁸O, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro GPR22 labeling and competition assays, compounds that incorporate ³H, ¹⁴C, ⁸²Br, ¹²⁵I, ¹³¹I, or ³⁵S may be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of ³H, ¹¹C, ¹⁸F, ¹⁴C, ¹²⁵I, ¹²⁴I, ¹³¹I, ³⁵S and ⁸²Br.

Synthetic methods for incorporating radio-isotopes into organic compounds are applicable to compounds of the invention and are well known in the art. These synthetic methods, for example, incorporating activity levels of tritium into target molecules, include, but are not limited to: catalytic reduction with tritium gas; reduction with sodium borohydride [³H]; reduction with Lithium Aluminum Hydride [³H]; tritium gas exposure labeling; and N-methylation using methyl iodide [³H].

Synthetic methods for incorporating activity levels of ¹²⁵I, into target molecules include: Sandmeyer and like reactions (see, e.g., Zhu et al., J. Org. Chem. 2002, 67:943-948, 202); ortho ¹²⁵Iodination of phenols (see, e.g., Collier et al., J. Labeled Compd. Radiopharm. 42:S264-S266, 1999); aryl and heteroaryl bromide exchange with ¹²⁵I (see, e.g., Bas et al., J. Labeled Compd Radiopharm. 44:S280-S282, 2001).

In some embodiments, a radioisotope-labeled version of a compound is identical to the compound, but for the addition of one or more substituents comprising a radionuclide. In some further embodiments, the compound is a polypeptide. In some further embodiments, the compound is an antibody or an antigen-binding fragment thereof. In some further embodiments, said antibody is monoclonal. The radionuclide that is incorporated in the instant radio-labeled compound will depend on the specific application of that radio-labeled compound.

Methods for adding one or more substituents comprising a radionuclide are within the purview of the skilled artisan and include, but are not limited to, addition of radioisotopic iodine by enzymatic method (Marchalonic, Biochem. J. 113:299-305, 1969; Thorell and Johansson, Biochim. et Biophys. Acta 251:363-369, 1969) and or by chloramine-T/iodogen/iodobead methods (Hunter and Greenwood, Nature 194:495-496, 1962; Greenwood et al., Biochem. J. 89:114-123, 1963).

Full-Length Cloning of Endogenous Human GPR22. Although a variety of expression vectors are available to those in the art, for purposes of utilization for both the endogenous and non-endogenous GPCRs, in some embodiments pCMV is used (American Type Culture Collection [ATCC] #203351. In some embodiments an adenoviral expression vector is used.

Recombinant DNA techniques relating to the subject matter of the present invention and well known to those of ordinary skill in the art can be found, e.g, in Maniatis T et al., Molecular Cloning: A Laboratory Manual (1989) Cold Spring Harbor Laboratory; U.S. Pat. No. 6,399,373; and PCT Application Number PCT/IB02/01461 published as WO 02/066505 on 29 Aug. 2002; the disclosure of each of which is hereby incorporated by reference in its entirety.

The human GPR22 cDNA is provided in the GenBank database, accession Number U66581. Full-length GPR22 may be cloned by PCR using primers such as, for example: 5′-TCCCCCGGGAAAAAAACCAACTGCTGCAAA-3′ (sense), 5′-TAGGATCCATTTGAATGTGGATTTGGTGAAA-3′ (antisense, containing a BamHI site) and using human genomic DNA as template. Amplification may be carried out using rTth polymerase (Perkin Elmer) with the buffer system provided by the manufacturer, 0.25 mM of each primer, and 0.2 mM of each 4 nucleotides. The cycle condition is 30 cycles of 94° C. for 1 min, 50° C. for 1 min and 72° C. for 1.5 min. The 5′ PCR primer is kinased and the 1.38 kb PCR fragment is digested with BamHI and cloned into EcoRV-BamHI site of pCMV expression vector.

Receptor Expression. Although a variety of cells are available to the art for the expression of proteins, it is most preferred that mammalian cells or melanophores be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells or melanophores. Of the mammalian cells, CHO, COS-7, 293 and 293T cells are particularly preferred, although the specific mammalian cell utilized can be predicated upon the particular needs of the artisan.

a. Transient transfection. On day one, 6×10⁶ per 10 cm dish of 293 cells well are plated out On day two, two reaction tubes are prepared (the proportions to follow for each tube are per plate): tube A is prepared by mixing 4 μg DNA (e.g., pCMV vector; pCMV vector with receptor cDNA, etc.) in 0.5 ml serum-free DMEM (Gibco BRL); tube B is prepared by mixing 24 μl lipofectamine (Gibco BRL) in 0.5 ml serum free DMEM. Tubes A and B are admixed by inversions (several times), followed by incubation at room temperature for 30-45 min. The admixture is referred to as the “transfection mixture”. Plated 293 cells are washed with 1×PBS, followed by addition of 5 ml serum free DMEM. 1 ml of the transfection mixture is added to the cells, followed by incubation for 4 hrs at 37° C./5% CO.sub.2. The transfection mixture is removed by aspiration, followed by the addition of 10 ml of DMEM/10% Fetal Bovine Serum Cells are incubated at 37° C./5% CO₂. After 48 hr incubation, cells are harvested and utilized for analysis.

b. Stable Cell Lines. Approximately 12×10⁶ 293 cells are plated on a 15 cm tissue culture plate and grown in DME High Glucose Medium containing ten percent fetal bovine serum and one percent sodium pyruvate, L-glutamine, and antibiotics. Twenty-four hours following plating of 293 cells (or to .about.80% confluency), the cells are transfected using 12 μg of DNA. The 12 μg of DNA is combined with 60 μl of lipofectamine and 2 mL of DME High Glucose Medium without serum. The medium is aspirated from the plates and the cells are washed once with medium without serum. The DNA, lipofectamine, and medium mixture are added to the plate along with 10 mL of medium without serum. Following incubation at 37° C. for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/mL. The transfected cells now undergo selection for positively transfected cells containing the G418 resistant gene. The medium is replaced every four to five days as selection occurs. During selection, cells are grown to create stable pools, or split for stable clonal selection.

Assays for Determination of GPCR Activation. A variety of approaches are available for assessment of activitation of human GPCRs. The following are illustrative; those of ordinary skill in the art are credited with the ability to determine those techniques that are preferentially beneficial for the needs of the artisan.

1. Membrane Binding Assays: ³⁵S-GTPγS Assay. When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, ³⁵S-GTPγS, can be utilized to demonstrate enhanced binding of ³⁵S-GTPγS to membranes expressing activated receptors. The advantage of using ³⁵S-GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate ³⁵S-GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The ³⁵S-GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM ³⁵S-GTPγS (this amount can be adjusted for optimization of results, although 1.2 is preferred) and 12.5 to 75 μg membrane protein (this amount can be adjusted for optimization) and 10 μM GDP (this amount can be changed for optimization) for 1 hour. Wheat germ agglutinin beads (25 μl; Amersham) are then added and the mixture incubated for another 30 minutes at room temperature. The tubes are then centrifuged at 1500×g for 5 minutes at room temperature and then counted in a scintillation counter.

2. Adenylyl Cyclase. A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 min. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer [comprising 1 μCi of tracer .sup.125I cAMP (50 μl) to 11 ml Detection Buffer] is prepared and maintained in accordance with the manufacturer's instructions. Assay Buffer is prepared fresh for screening and contained 50 μl of Stimulation Buffer, 3 μl of test compound (12 μM final assay concentration) and 50 μl cells, Assay Buffer is stored on ice until utilized. The assay is initiated by addition of 50 μl of cAMP standards to appropriate wells followed by addition of 50 μl of PBSA to wells H-11 and H12. 50 μl of Stimulation Buffer is added to all wells. DMSO (or selected candidate compounds) was added to appropriate wells using a pin tool capable of dispensing 3 μl of compound solution, with a final assay concentration of 12 μM test compound and 10 μl total assay volume. The cells are then added to the wells and incubated for 60 min at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which was contained within each assay plate.

3. Cell-Based cAMP for Gi Coupled Target GPCRs. TSHR is a Gs-coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gi coupled receptor can be accomplished by co-transfecting, most preferably, non-endogenous, constitutively activated TSHR (TSHR-A6231) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. Such approach may be used to effectively generate a signal when a cAMP assay is used; this approach is preferably used in the direct identification of candidate compounds against Gi coupled receptors. It is noted that for a Gi coupled GPCR, when this approach is used, an antagonist or inverse agonist of the target GPCR will increase the cAMP signal and an agonist will decrease the cAMP signal.

On day one, 2×10⁴ 293 cells per well are plated out. On day two, two reaction tubes are prepared (the proportions to follow for each tube are per plate): tube A is prepared by mixing 2 μg DNA of each receptor transfected into the mammalian cells, for a total of 4 μg DNA [e.g., pCMV vector; pCMV vector with mutated TSHR (TSHR-A623I); TSHR-A6231 and GPCR, etc.] in 1.2 ml serum free DMEM (Irvine Scientific, Irvine, Calif.); tube B is prepared by mixing 120 μl lipofectamine (Gibco BRL) in 1.2 ml serum free DMEM. Tubes A and B are then be admixed by inversions (several times), followed by incubation at room temperature for 30-45 min. The admixture is referred to as the “transfection mixture”. Plated 293 cells are washed with 1×PBS, followed by addition of 10 ml serum free DMEM. 2.4 ml of the transfection mixture are then added to the cells, followed by incubation for 4 hrs at 37° C./5% CO₂. The transfection mixture is then removed by aspiration, followed by the addition of 25 ml of DMEM/10% Fetal Bovine Serum. Cells are then incubated at 37° C./5% CO₂. After 24 hr incubation, cells are harvested and utilized for analysis.

A Flash Plate™ Adenylyl Cyclase kit (New England Nuclear; Cat. No. SMP004A) is designed for cell-based assays, but it can be modified for use with crude plasma membranes. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS is added to each plate. Cells are pipetted off the plate and the cell suspension is collected into a 50 ml conical centrifuge tube. Cells are then centrifuged at room temperature at 1,100 rpm for 5 min. The cell pellet is carefully resuspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer ¹²⁵I cAMP (50μ)) to 11 ml Detection Buffer) is prepared and maintained in accordance with the manufacturer's instructions. Assay Buffer should be prepared fresh for screening and contains 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer is added to all wells. Selected compounds (e.g., TSH) are added to appropriate wells using a pin tool capable of dispensing 3 μl of compound solution, with a final assay concentration of 12 μM test compound and 100 μl total assay volume. The cells are then added to the wells and incubated for 60 min at room temperature. 100 μl of Detection Mix containing tracer cAMP is then added to the wells. Plates are then incubated for an additional 2 hours followed by counting in a Wallac MicroBeta scintillation counter. Values of cAMP/well are then extrapolated from a standard cAMP curve which is contained within each assay plate.

4. Reporter-Based Assays

a. CRE-Luc Reporter Assay (Gs-Associated Receptors). 293 and 293T cells are plated-out on 96 well plates at a density of 2×10⁴ cells per well and are transfected using Lipofectamine Reagent (BRL) the following day according to manufacturer instructions. A DNA/lipid mixture is prepared for each 6-well transfection as follows: 260 ng of plasmid DNA in 100 μl of DMEM were gently mixed with 2 μl of lipid in 100 μl of DMEM (the 260 ng of plasmid DNA consisted of 200 ng of a 8.times.CRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8×CRE-Luc reporter plasmid was prepared as follows: vector SRIF-β-gal was obtained by cloning the rat somatostatin promoter (−71/+51) at BgIV-HindIII site in the pβ-gal-Basic Vector (Clontech). Eight (8) copies of cAMP response element were obtained by PCR from an adenovirus template AdpCF126CCRE8 (see, 7 Human Gene Therapy 1883 (1996)) and cloned into the SRIF β-gal vector at the Kpn-BglV site, resulting in the 8.times.CRE-.beta.-gal reporter vector. The 8×CRE-Luc reporter plasmid was generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture is diluted with 40 μl of DMEM and 100 μl of the diluted mixture was added to each well. 100 μl of DMEM with 10% FCS are added to each well after a four hr incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors). A method to detect Gq stimulation depends on the known property of Gq-dependent phospholipase C to cause the activation of genes containing AP1 elements in their promoter. A Pathdetect™ AP-1 cis-Reporting System (Stratagene, Catalogue # 219073) can be utilized following the protocol set forth above with respect to the CREB reporter assay, except that the components of the calcium phosphate precipitate are 410 ng pAP1-Luc, 80 ng pCMV-receptor expression plasmid, and 20 ng CMV-SEAP.

c. SRF-Luc Reporter Assay (Gq-Associated Receptors). One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with 1 μM Angiotensin, where indicated. Cells are then lysed and assayed for luciferase activity using a Luclite™ Kit (Packard, Cat. # 6016911) and Trilux 1450 Microbeta liquid scintillation and luminescence counter (Wallac) as per the manufacturer's instructions. The data can be analyzed using GraphPad Prism™ 2.0a (GraphPad Software Inc.)

Intracellular IP3 Accumulation Assay (Gq-Associated Receptors). On day 1, cells comprising the receptors (endogenous and/or non-endogenous) can be plated onto 24 well plates, usually 1×10⁵ cells/well (although his umber can be optimized. On day 2 cells can be transfected by firstly mixing 0.25 μg DNA in 50 μl serum free DMEM/well and 2 μl lipofectamine in 50 μl serum-free DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of .sup.3H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10 xx ketanserin (ket) to final concentration of 10 μM. The cells are then incubated for 30 min at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added per tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

Fusion Protein Preparation. Standard recombinant DNA techniques are used to create a “universal” G protein vectors, preferably with the sequence for the GPCR upstream and in-frame with that of the G protein. See, e.g., U.S. patent application no. 20070224127 for one such example.

### Protocol: Direct Identification of Antagonists

A. ³⁵-GTPγS Assay. In some embodiments, an endogenous GPCR is utilized for the direct identification of candidate compounds as, e.g., agonists or antagonists. In some embodiments, an endogenous constitutively active GPCR or a non-endogenous constitutively activated GPCR is utilized for the direct identification of candidate compounds as, e.g., inverse agonists or agonists. In some embodiments, a GPCR Fusion Protein comprising an endogenous, constitutively active GPCR or a non-endogenous constitutively activated GPCR is utilized for the direct identification of candidate compounds as, e.g, antagonists. In said embodiments, the following assay protocols are provided for said direct identification.

In some embodiments membranes comprising the GPCR/Fusion Protein of interest and for use in the direct identification of candidate compounds are preferably prepared as follows:

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl.sub.2, pH 7.4.

All materials are kept on ice throughout the procedure. Firstly, the media are aspirated from a confluent monolayer of cells, followed by rinse with 10 ml cold PBS, followed by aspiration. Thereafter, 5 ml of Membrane Scrape Buffer is added to scrape cells; this is followed by transfer of cellular extract into 50 ml centrifuge tubes (centrifuged at 20,000 rpm for 17 minutes at 4° C.). Thereafter, the supernatant is aspirated and the pellet is resuspended in 30 ml Membrane Wash Buffer followed by centrifuge at 20,000 rpm for 17 minutes at 4° C. The supernatant is then aspirated and the pellet resuspended in Binding Buffer. This will then be homogenized using a Brinkman Polytron™ homogenizer (15-20 second bursts until the all material is in suspension). This is referred to herein as “Membrane Protein”.

Following the homogenization, protein concentration of the membranes is determined using the Bradford Protein Assay (protein can be diluted to about 1.5 mg/ml, aliquoted and frozen (−80° C.) for later use; when frozen, protocol for use is as follows: on the day of the assay, frozen Membrane Protein is thawed at room temperature, followed by vortex and then homogenized with a Polytron at about 12×1,000 rpm for about 5-10 seconds; for multiple preparations, the homogenizer should be thoroughly cleaned between homogenization of different preparations). Duplicate tubes are prepared, one including the membrane, and one as a control “blank”. Each contains 800 μl Binding Buffer. Thereafter, 10 μl of Bradford Protein Standard (1 mg/ml) is added to each tube, and 10 μl of membrane Protein is then added to just one tube (not the blank). Thereafter, 200 μl of Bradford Dye Reagent is added to each tube, followed by vortex of each. After five (5) minutes, the tube is re-vortexed and the material therein is transferred to cuvettes. The cuvettes will then be read using a CECIL 3041 spectrophotometer, at wavelength 595.

Direct Identification Assay. GDP Buffer consists of 37.5 ml Binding Buffer and 2 mg GDP (Sigma, cat. no. G-7127), followed by a series of dilutions in Binding Buffer to obtain 0.2 μM GDP (final concentration of GDP in each well was 0.1 μM GDP); each well comprising a candidate compound, has a final volume of 200 μl consisting of 100 μl GDP Buffer (final concentration, 0.1 μM GDP), 50 μl Membrane Protein in Binding Buffer, and 50 μl ³⁵S-GTPγS (0.6 nM) in Binding Buffer (2.5 μl ³⁵S-GTPγS per 10 ml Binding Buffer).

Candidate compounds are preferably screened using a 96-well plate format (these can be frozen at −80° C.). Membrane Protein (or membranes with expression vector excluding the GPCR Fusion Protein, as control), is homogenized briefly until in suspension. Protein concentration is then determined using the Bradford Protein Assay. Membrane Protein (and control) is then diluted to 0.25 mg/ml in Binding Buffer (final assay concentration, 12.5 μg/well). Thereafter, 100 μl GDP Buffer was added to each well of a Wallac Scintistrip™ (Wallac). A 5 μl pin-tool will then be used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×), and excess liquid should be shaken from the tool after each rinse and dried with paper and kimwipes. Thereafter, 50 μl of Membrane Protein is added to each well (a control well comprising membranes without the GPCR Fusion Protein is also utilized), and pre-incubated for 5-10 minutes at room temperature. Thereafter, 50 μl of ³⁵S-GTPγS (0.6 nM) in Binding Buffer is added to each well, followed by incubation on a shaker for 60 minutes at room temperature (again, in this example, plates are covered with foil). The assay is then stopped by spinning of the plates at 4000 RPM for 15 minutes at 22° C. The plates are then aspirated with an 8 channel manifold and sealed with plate covers. The plates are then read on a Wallac 1450 using setting “Prot. #37” (as per manufacturer instructions).

B. Cyclic AMP Assay. Another assay approach for directly identifying candidate compounds as, e.g., antagonists, is accomplished by utilizing a cyclase-based assay. In addition to direct identification, this assay approach can be utilized as an independent approach to provide confirmation of the results from the ³⁵S-GTPγS approach as set forth above.

A modified Flash Plate™ Adenylyl Cyclase kit (New England Nuclear; Cat. No. SMP004A) is preferably utilized for direct identification of candidate compounds antagonists to endogenous or constitutively active GPCRs in accordance with the following protocol.

Transfected cells are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000×g for 15 minutes at 4° C. The resulting pellet is then resuspended in buffer containing 20 mM HEPES, pH 7.4 and 0.1 mM EDTA, homogenized for 10 seconds, followed by centrifugation at 49,000×g for 15 minutes at 4° C. The resulting pellet is then stored at −80° C. until utilized. On the day of direct identification screening, the membrane pellet is slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use).

cAMP standards and Detection Buffer [comprising 2 μCi of tracer ¹²⁵I cAMP (100 μl) to 11 ml Detection Buffer] are prepared and maintained in accordance with the manufacturer's instructions. Assay Buffer is prepared fresh for screening and contained 20 mM HEPES, pH 7.4, 10 mM MgCl₂, 20 mM phosphocreatine (Sigma), 0.1 units/ml creatine phosphokinase (Sigma), 50 μM GTP (Sigma), and 0.2 mM ATP (Sigma). Assay Buffer is then stored on ice until utilized.

Candidate compounds (if frozen, thaw at room temperature) are added, preferably, to 96-well plate wells (3 μl/well; 12 μM final assay concentration), together with 40 μl Membrane Protein (30 μg/well) and 50 μl of Assay Buffer. This admixture is then incubated for 30 minutes at room temperature, with gentle shaking.

Following the incubation, 10 μl of Detection Buffer is added to each well, followed by incubation for 2-24 hours. Plates are then counted in a Wallac MicroBeta™ plate reader using “Prot. #31” (as per manufacturer instructions).

By focusing on assay techniques that are based upon receptor function, and not compound binding affinity, we are able to ascertain compounds that are able to reduce the functional activity of this receptor as well as increase the functional activity of the receptor.

Fluorometric Imaging Plate Reader (FLIPR) Assay for the Measurement of Intracellular Calcium Concentration. Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 min.

After the 1 hr incubation, the Fluo4-AM incubation buffer is removed and the cells are washed 2× with 100 μl wash buffer. In each well is left 100 μl wash buffer. The plate is returned to the incubator at 37° C./5% CO₂ for 60 min.

FLIPR (Fluorometric Imaging Plate Reader; Molecular Device) is programmed to add 50 μl candidate compound on the 30th second and to record transient changes in intracellular calcium concentration (Ca²⁺) evoked by the candidate compound for another 150 seconds. Total fluorescence change counts are used to determine antagonist activity using the FLIPR software. The instrument software normalizes the fluorescent reading to give equivalent initial readings at zero.

In some embodiments, the cells comprising Target Receptor further comprise promiscuous G alpha 15/16 or the chimeric Gq/Gi alpha unit.

Although the foregoing provides a FLIPR assay for antagonist activity using stably transfected cells, a person of ordinary skill in the art would readily be able to modify the assay in order to characterize antagonist activity. Alternatively, transiently transfected cells could be used.

Melanophore Technology. Melanophores are skin cells found in lower vertebrates. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a Gi-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this re-dispersion. In addition, the technology is also suited to the study of certain receptor tyrosine kinases. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, against GPCRs. This method can be conducted by introducing test cells of a pigment cell line capable of dispersing or aggregating their pigment in response to a specific stimulus and expressing an exogenous clone coding for the GCPR. A stimulant, e.g., melatonin, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light.

Materials and methods are followed according to the disclosure of U.S. Pat. No. 5,462,856 and U.S. Pat. No. 6,051,386. These patent disclosures are hereby incorporated by reference in their entirety.

The cells are plated in 96-well plates (one receptor per plate). 48 hours post-transfection, half of the cells on each plate are treated with 10 nM melatonin. Melatonin activates an endogenous Gi-coupled receptor in the melanophores and causes them to aggregate their pigment. The remaining half of the cells are transferred to serum-free medium 0.7×L-15 (Gibco). After one hour, the cells in serum-free media remain in a pigment-dispersed state while the melatonin-treated cells are in a pigment-aggregated state. At this point, the cells are treated with a dose response of a candidate compound. If the plated GPCRs bind to the candidate compound, the melanophores would be expected to undergo a color change in response to the compound.

Cardioprotection. A modulator of the invention can be shown to be cardioprotective using the in vivo rat model of Fryer et al. (Circ. Res. 84:846-851, 1999). Said modulator is administered by intraperitoneal injection. Preferred dose is 0.1-100 mg/kg. Other preferred dose is selected from the group consisting of: 0.1 mg/kg, 0.3 mg/kg; 1.0 mg/kg; 3.0 mg/kg; 10 mg/kg; 30 mg/kg and 100 mg/kg. The placebo group is administered vehicle alone.

Male Wistar rats, 350 to 450 g, are used for all phases of this study. Rats are administered said modulator or saline 1, 12, 24, 48, or 72 hours before the surgical protocol through intraperitoneal injection. Subsequently, rats are anesthetized via intraperitoneal administration of thiobutabarbital sodium (Inactin, Research Biochemical International; 100 mg/kg). A tracheotomy is performed, and the trachea is intubated with a cannula connected to a rodent ventilator (model CIV-101, Columbus Instruments, or model 683, Harvard Apparatus). Rats are ventilated with room air supplemented with O₂ at 60-65 breaths per minute. Atelectasis is prevented by maintaining a positive end-expiratory pressure of 5 to 10 mm H₂O. Arterial pH, P_{CO2}, and P_{CO2} are monitored at control, 15 minutes of occlusion, and 60 and 120 minutes of reperfusion by a blood gas system (AVL 995 pH/blood gas analyzer, AVL Medical Instruments) and maintained within a normal physiological range (pH 7.35 to 7.45; P_{CO2} 25 to 40 mm Hg; and P_{O2} 80 to 110 mm Hg) by adjusting the respiratory rate and/or tidal volume. Body temperature is maintained at 38° C. by the use of a heating pad, and bicarbonate is administered intravenously as needed to maintain arterial blood pH within normal physiological levels.

The right carotid artery is cannulated to measure blood pressure and heart rate via a Gould PE50 or Gould PE23 pressure transducer connected to a Grass (model 7) polygraph. The right jugular vein is cannulated for saline, bicarbonate, and drug infusion. A left thoracotomy is performed at the fifth intercostals space followed by a pericardiotomy and adjustment of the left atrial appendage to reveal the location of the left coronary artery. A ligature (6-0 prolene) is passed below the coronary artery from the area immediately below the left atrial appendage to the right portion of the left ventricle. The ends of the suture are threaded through a propylene tube to form a snare. The coronary artery is occluded by pulling the ends of the suture taut and clamping the snare onto the epicaridal surface with a hemostat. Coronary artery occlusion is verified by epicardial cyanosis and a subsequent decrease in blood pressure. Reperfusion of the heart is initiated via unclamping the hemostat and loosening the snare and is confirmed by visualizing an epicardial hyperemic response. Heart rate and blood pressure are allowed to stabilize before the experimental protocols are initiated.

Rats are randomly divided into the designated experimental groups. Control rats are administered saline 24 hours before 30 minutes of regional ischemia and 2 hours of reperfusion (I/R). To show acute cardioprotection induced by said modulator, said modulator is administered 1 hour before a prolonged ischemic insult. To show the delayed cardioprotection against an acute ischemic insult, said modulator is administered at the designated doses either 12 or 24 hours before I/R. Said modulator is also administered at the designated doses either 48 or 72 hours before I/R.

On completion of the above protocols, the coronary artery is occluded, and the area at risk (AAR) is determined by negative staining with patent blue dye administered via the jugular vein. The rat is euthanized with a 15% KCl solution. The heart is excised and the left ventricle is dissected from the remaining tissue and subsequently cut into six thin, cross-sectional pieces. This allows for the delineation of the normal area, stained blue, versus the AAR, which subsequently remained pink. The AAR is excised from the nonischemic area, and the tissues are placed in separate vials and incubated for 15 minutes with 1.0% 2,3,5-triphenyltetrazolium chloride (TTC) stain in 100 mmol/L phosphate buffer (pH 7.4) at 37° C. TTC is an indicator of viable and nonviable tissue. TTC is reduced by dehydrogenase enzymes present in viable myocardium and results in a formazan precipitate, which induces a deep red color, whereas the infarcted area remains gray (Klein et al., Virchows Arch. [Pathol. Anat.] 393:287-97, 1981). Tissues are stored in vials of 10% formaldehyde overnight, and the infracted myocardium is dissected from the AAR under the illumination of a dissecting microscope (Cambridge Instruments). Infarct size (IS), AAR, and left ventricular weight (LV) are determined by gravimetric analysis. AAR is expressed as a percentage of the LV (AAR/LV), and IS is expressed as a percentage of the AAR (IS/AAR).

Rats are excluded from data analysis if they exhibit severe hypotension (<30 mm Hg systolic blood pressure) or if adequate blood gas values within a normal physiological range are unable to be maintained because of metabolic acidosis or alkalosis.

All values are expressed as mean±SEM. One-way ANOVA with Bonferroni's test is used to determine whether any significant differences exist among groups for hemodynamics, IS, and AAR. Significant differences are determined at P<0.05. A reduction of IS/AAR is indicative of cardioprotection.

Oral Bioavailability. Physico-chemico analytical approaches for directly assessing oral bioavailability are well known to those of ordinary skill in the art and may be used (see, e.g., without limitation: Wong et al., Cardiovasc. Drug Rev. 20:137-152, 2002; and Buchan et al., Headache Suppl 2:S54-S62, 2002). By way of further illustration and not limitation, said alternative analytical approaches may comprise liquid chromatography-tandem mass spectrometry (Chavez-Eng et al., J. Chromatogr B Analyt. Technol. Biomed. Life Sci. 767:117-129, 2002; Jetter et al., Clin. Pharmacol. Ther. 71:21-29, 2002; Zimmerman et al., J. Clin. Pharmacol. 39:1155-1161, 1999; and Barrish et al., Rapid Commun. Mass Spectrom. 10:1033-1037, 1996).

Positron emission tomography (PET) has been successfully used to obtain direct measurements of drug distribution, including oral bioavailability, in the mammalian body following oral administration of the drug (Gulyas et al., Eur. J. Nucl. Med. Mol. Imaging. (2002) 29:1031-1038).

Alternatively, oral bioavailability of a modulator of the invention may be determined on the basis of in vivo data developed, as for example by way of illustration and not limitation through the rat model of Example 18. The modulator is administered by oral gavage at doses ranging from 0.1 mg kg⁻¹ to 100 mg kg⁻¹. Oral administration of the modulator is shown to confer cardioprotection. The effect of the modulator is shown to be dose-dependent and comparable to the effect after intraperitoneal administration. The dose of modulator required to achieve half-maximal reduction of IS/AAR through oral administration is compared to the dose of modulator required to achieve half-maximal reduction of IS/AAR through intraperitoneal administration. By way of illustration, if said oral dose is twice said intraperitoneal dose, then the oral bioavailability of the modulator is taken to be 50%. More generally, if said oral dose is y mg kg⁻¹ and said intraperitoneal dose is z mg kg⁻¹, then the oral bioavailability of the modulator as a percentage is taken to be [(z/y)×100].

It would be readily apparent to anyone of ordinary skill in the art that a determination of oral bioavailability of a modulator of the invention can be carried out using an in vivo animal model other than the one presented here for purposes of illustration and not limitation. It would also be readily apparent to anyone of ordinary skill in the art that the bioactivity readout for said oral bioavailability could be a parameter other than IS/AAR. It is readily envisioned that the reference route of administration may be other than intraperitoneal. In some embodiments, said reference route of administration may be intravenous.

In some embodiments, oral bioavailability of a modulator of the invention is at least 1%, at least 5%, at least 10%, at least 15%, at least 20%, at least 25%, at least 30%, at least 35%, at least 40%, or at least 45% relative to intraperitoneal injection.

Transgenic Mouse/Rat/Pig Comprising Expression of a Human GPR22 GPCR. Methods of making transgenic animals such as mice, rats, and pigs are well known to those of ordinary skill in the art, and any such method can be used in the present invention. Briefly, transgenic mammals can be produced, e.g., by transfecting a pluripotential stem cell such as an ES cell with a polynucleotide (“transgene”) encoding a human GPR22GPCR. Successfully transformed ES cells can then be introduced into an early stage embryo that is then implanted into the uterus of a mammal of the same species. In certain cases, the transformed (“transgenic”) cells will comprise part of the germ line of the resulting animal and adult animals comprising the transgenic cells in the germ line can then be mated to other animals, thereby eventually producing a population of transgenic animals that have the transgene in each of their cells and that can stably transmit the transgene to each of their offspring. Other methods of introducing the polynucleotide can be used, for example introducing the polynucleotide encoding a human GPR22GPCR into a fertilized egg or early stage embryo via microinjection. Alternatively, the transgene may be introduced into an animal by infection of zygotes with a retrovirus containing the transgene (Jaenisch, Proc. Natl. Acad. Sci. USA 73:1260-1264, 1976). Methods of making transgenic mammals are described, e.g., in: Wall et al., J. Cell. Biochem. 49:113-120, 1992; Hogan et al., in Manipulating the Mouse Embryo. A Laboratory Manual., Cold Spring Harbor Laboratory Press, Cold Spring Harbor, N.Y., 1986; Costa et al., FASEB J. 13:1762-1773, 1999; WO 91/08216; U.S. Pat. No. 4,736,866; and U.S. Pat. No. 6,504,080.

In some embodiments, said expression of a human GPR22GPCR is cardiomyocyte-selective. In some embodiments, said cardiomyocyte-selective expression of said human GPR22GPCR is conferred by alpha myosin heavy chain promoter (Subramaniam et al., J. Biol. Chem. 266:24613-24620, 1991).

Transgenic in vivo Animal Model of Cardioprotection. A compound of the present invention can be shown to have efficacy for cardioprotection using a transgenic in vivo animal model described above. In some embodiments, said animal is mouse, rat or pig.

Said compound can be assessed for efficacy for cardioprotection by administering said compound to said transgenic animal and determining if said administration leads to a reduction in IS/AAR in an in vivo rat model or an in vivo model in mouse or pig analogous thereto relative to said transgenic animal administered vehicle alone.

In preferred embodiments, said compound is modulator of the invention. In some embodiments, said modulator lowers the intracellular level of cAMP. In some embodiments, said modulator is an antagonist. Preferred dose is 0.1-100 mg/kg. Other preferred dose is selected from the group consisting of: 0.1 mg/kg, 0.3 mg/kg; 1.0 mg/kg; 3.0 mg/kg; 10 mg/kg; 30 mg/kg and 100 mg/kg. The placebo group is administered vehicle alone. In some embodiments, said dose is administered daily. In some embodiments, said dose is administered for a period selected from the group of one week, two weeks, three weeks, and four weeks. It is noted that this route of administration, these dosage ranges, this frequency of dose administration, and this duration of dose administration are intended to be illustrative and not limiting to the invention.

Mouse Comprising Knockout of GPR22Gene. A preferred DNA construct will comprise, from 5′-end to 3′-end: (a) a first nucleotide sequence that is comprised in the mouse GPR22 genomic sequence; (b) a nucleotide sequence comprising a positive selection marker, such as the marker for neomycin resistance (neo); and (c) a second nucleotide sequence that is comprised in the mouse GPR22 genomic sequence and is located on the genome downstream of the first mouse GPR22 nucleotide sequence (a). Mouse GPR22 genomic sequence is isolated using methods well known to those of ordinary skill in the art. Probes for said isolation of mouse GPR22 genomic sequence is derived from cDNA encoding a mouse GPR22 polypeptide, wherein said cDNA may be obtained using as template mRNA from mouse heart, lung, or adipose tissue.

In preferred embodiments, this DNA construct also comprises a negative selection marker located upstream the nucleotide sequence (a) or downstream the nucleotide sequence (c). Preferably, the negative selection marker comprises the thymidine kinase (tk) gene (Thomas et al., Cell 44:419-428, 1986), the hygromycin beta gene (Te Riele et al., Nature 348:649-651, 1990], the hprt gene (Van der Lugt et al., Gene 105:263-267, 1991; Reid et al., Proc. Natl. Acad. Sci. USA 87:4299-4303, 1990) or the Diptheria toxin A fragment (Dt-A) gene (Nada et al., Cell 73:1125-1135, 1993; Yagi et al., Proc. Natl. Acad. Sci. USA 87:9918-9922, 1990). Preferably, the positive selection marker is located within a mouse GPR22 exon sequence so as to interrupt the sequence encoding a mouse GPR22 polypeptide. These replacement vectors are described, for example, by Thomas et al., Cell 44:419-428, 1986; Thomas et al., Cell 51:503-512, 1987; Mansour et al., Nature 336:348-352, 1988; Koller et al., Ann. Rev. Immunol. 10:705-730, 1992; and U.S. Pat. No. 5,631,153.

The first and second nucleotide sequences (a) and (c) may be indifferently located within a mouse GPR22 regulatory sequence, an intronic sequence, an exon sequence or a sequence containing both regulatory and/or intronic and/or exon sequences. The size of the nucleotide sequences (a) and (c) ranges from 1 to 50 kb, preferably from 1 to 10 kb, more preferably from 2 to 6 kb, and most preferably from 2 to 4 kb.

Methods of making a mouse comprising knockout of a selected gene are well known to those of ordinary skill in the art and have been used to successfully inactivate a wide range of genes.

Rat Comprising Knockout of GPR22Gene. Gene targeting technology for the rat is less robust than that for the mouse and is an area of active interest One approach is to inactivate rat GPR22 gene in rat embryonic stem cell (ESC)-like cells and then inject cells with inactivated rat GPR22 gene into rat blastocysts generated after fusion of two-cell embryos (Krivokharchenko et al., Mol. Reprod. Dev. 61:460-465, 2002).

The rat gene is identified by screening a rat genomic library under stringent hybridization conditions using the rat GPR22 polynucleotide. Full-length or essentially fill-length rat GPR22 cDNA is identified by screening a rat heart or brain cDNA library under similar conditions. Conditions for stringent nucleic acid hybridization are well known to persons of ordinary skill in the art (Maniatis et al., Molecular Cloning: A Laboratory Manual, Cold Spring Harbor, N.Y., 1982).

An alternative approach is to inactivate rat GPR22 gene in rat ESC-like cells and then transfer the nucleus of the rat ESC-like cells having inactivated rat GPR22 gene into enucleated oocytes (Sato et al., Hum. Cell 14:301-304, 2001; Wakayama and Yanagimachi, Semin. Cell Dev. Biol. 10:253-258, 1999; Hochedlinger and Jaenisch, Nature 415:1035-1038, 2002; Yanagimachi, Mol. Cell. Endocrinol. 187:241-248, 2002).

Methods analogous or alternative (also see, e.g., Zan et al, Nature Biotech. 21:645-651, 2003) to those described for the mouse may be used to make a rat comprising knockout of GPR22 gene.

Pig Comprising Knockout of GPR22Gene. Analogous or alternative methods may be used to make a pig comprising knockout of GPR22 gene (see, e.g., Lai et al., Science 295:1089-1092, 2002).

Mouse Comprising a Cardiomyocyte-Selective Knockout of GPR22Gene. These new DNA constructs make use of the site specific recombination system of the P1 phage. The P1 phage possesses a recombinase called Cre that interacts with a 34 base pair loxP site. The loxP site is composed of two palindromic sequences of 13 bp separated by an 8 bp conserved sequence (Hoess et al, Nucl. Acids Res. 14:2287-2300, 1986). The recombination by the Cre enzyme between two loxP sites having an identical orientation leads to the deletion of the DNA fragment.

The Cre-loxP system used in combination with a homologous recombination technique has been first described by Gu et al., Cell 73:1155-1164, 1993; and Gu et al., Science 265:103-106, 1994]. Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be brought at the desired time either by (a) incubating the recombinant cell hosts in a culture medium containing this enzyme, by injecting the Cre enzyme directly into the desired cell, such as by lipofection of the enzyme into the cells (such as described by Baubonis and Sauer, Nucl. Acids Res. 21:2025-2029, 1993); (b) transfecting the cell host with a vector comprising the Cre coding sequence operably linked to a promoter functional in the recombinant cell host, which promoter being optionally inducible, said vector being introduced in the recombinant cell host (such as described by Gu et al., Cell 73:1155-1164, 1993; and Sauer and Henderson, Proc. Natl. Acad. Sci. USA 85:5166-5170, 1988); (c) introducing into the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al., Science 265:103-106, 1994.

Vectors and methods using the Cre-loxP system are described, e.g., by Zou et al. (1994); Minamisawa et al., J. Biol. Chem. 274:10066-10070, 1999; Chen et al., J. Biol. Chem. 273:1252-1256, 1998; Chen et al., Development 125:1943-1949, 1998.

In preferred embodiments of the invention, Cre is introduced into the genome of the cell host by strategy (c) above, wherein said promoter is cardiomyocyte selective and leads to cardiomyocyte-selective disruption of (loxP-flanked; “floxed”) mouse GPR22 genomic sequence. In some embodiments, said cardiomyocyte-selective promoter is that for the ventricular specific isoform of myosin light chain 2 (mlc-2v) (Minamisawa et al., J. Biol. Chem. 274:10066-10070, 1999; Chen et al., J. Biol. Chem. 273:1252-1256, 1998). Transgenic mice comprising insertion of Cre recombinase coding sequence into the endogenous mlc-2v locus (“mlc-2v cre knock-in mice”) have been described (Chen et al., Development 125:1943-1949, 1998). Methods for floxing a selected gene are within the purview of those of ordinary skill in the art (see, e.g., Chen et al., Development 125:1943-1049, 1998).

In some embodiments, the invention features a method of making a mouse comprising a cardiomyocyte-selective knockout of GPR22 gene, comprising crossing the mlc-2 cre allele, supra, with a floxed GPR22 gene.

Other methods of making a mouse comprising a cardiomyocyte-selective knockout of GPR22 gene are well known to persons of ordinary skill in the art; see, e.g, Kuhn and Torres, Meth. Mol. Biol. 180:175-204, 2002; Sauer, Methods 14:381-92, 1998; Gutstein et al., Circulation Res. 88:333, 2001; Minamino et al., Circulation Research 88:587, 2001; and Bex et al., J. Urol. 168:2641-2644, 2002.

Rat Comprising a Cardiomyocyte-Selective Knockout of GPR22 Gene. Analogous or alternative methods may be used to make a rat comprising a cardiomyocyte knockout of GPR22 gene (see, e.g., Zan et al, Nature Biotechnol. 21:645-651, 2003).

Pig Comprising a Cardiomyocyte-Selective Knockout of GPR22 Gene. Analogous or alternative methods may be used to make a pig comprising a cardiomyocyte-selective knockout of GPR22 gene (see, e.g., Lai et al., Science 295:1089-1092, 2002).

Other uses of the disclosed receptors and methods will become apparent to those in the art based upon, inter alia, a review of this patent document.

The invention will be better understood by reference to the following Examples, which are intended to merely illustrate the best mode now known for practicing the invention. The scope of the invention is not to be considered limited thereto.

### EXAMPLE 1

### Ablation of Retinoid Signaling in the Myocardium

Heart failure upon retinoid signaling ablation. Vitamin A serves a crucial role during development and in homeostasis. Nutritional deficiency or excess retinoic acid both lead to embryonic malformations depending on the time of exposure and dose (Dickman et al., Development 124:3111-3121, 1997). Retinoids exert their functions by binding to nuclear receptors, RAR and RXR, both of which are members of the super-family of ligand-inducible transcriptional regulators (Evans, Science 240:889-895, 1988; Green and Chambon, Trends Genet. 4:309-314, 1988). The ligand-activated receptors differentially regulate gene expression through specific hormone-responsive DNA elements, where the receptors can bind as heterodimers or homodimers (Umesono et al., Cell 65:1255-1266, 1991).

Several lines of evidence suggest a role for retinoid signaling in adult myocytes. Over-expression of a truncated RXRα in transgenic mice resulted in low penetrance cardiac hypertrophy (Subbarayan et al., J. Clin. Invest. 105:387-394, 2000), and the expression in cardiomyocytes of a RAR-βGal fusion protein induced dilated cardiomayopathy in transgenic mice (Colbert et al., J. Clin. Invest. 100:1958-1968, 1997).

In order to examine the requirement for retinoid signaling in the adult myocytes, we used a tissue-specific inducible dominant negative RAR (RAR 403) that was previously used to evaluate retinoid function during development (Damm et al., Proc. Natl. Acad. Sci. USA 90:2989-2993, 1993). RAR 403 is a transcriptional inactive dominant-negative RAR by deletion of helix 12 (Damm et al., Proc. Natl. Acad. Sci. USA 90:2989-2993, 1993). When RAR403 is paired with wild-type RXR, both subunits of the RXR-RAR403 heterodimer are resistant to agonist-dependent activation (Osburn et al., Mol. Cell. Biol. 21:4909-4918, 2001).

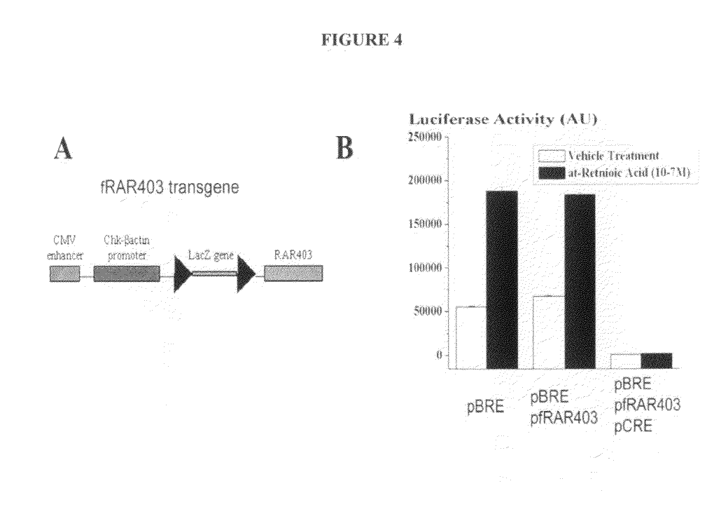

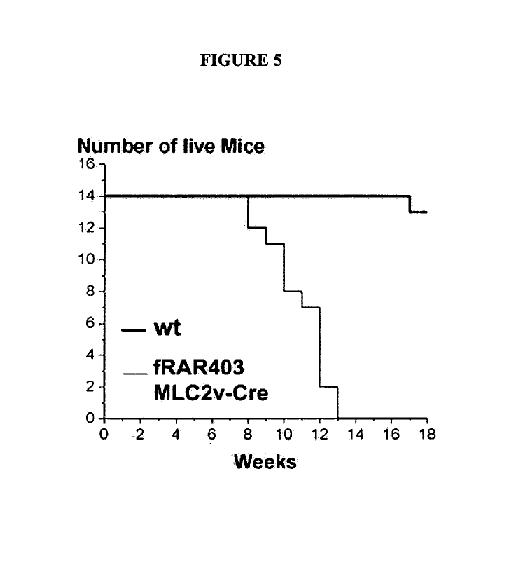

We obtained transgenic mice that express a dominant negative form of the RAR receptor (RAR 403) in a Cre-inducible allele, fRAR403 (FIG. 4A). Uninduced transgenic mice breed normally and have normal life expectancy. Northern analysis indicated that RAR403 is only expressed in the presence of Cre-recombinase expression.

In order to obliterate the signaling through retinoids (RAR and RXR) in ventricular myocytes, we crossed the inducible fRAR403 mice with a transgenic mouse containing Cre knocked into the MLC-2v locus. MLC-2V-cre mice have been studied comprehensively and demonstrate efficient and selective expression of Cre in ventricular myocytes (Chen et al., Development 125:1943-1949, 1998). Transgenic mice hemizygous for MLC-2V are phenotypically normal and express Cre efficiently and restricted to ventricular myocytes starting as early as embryonic day 8 (Chen et al., Development 125:1943-1949, 1998).

In early postnatal stages, fRAR403:MLC2vCre mice lived to weaning age and were indistinguishable from all littermates (dnRAR403 mice, MLC-2V-cre transgenic mice or wild type). However, between weeks 8 and 13 after birth, fRAR403:MLC2vCre mice were found with massive fluid accumulation in the abdomen and chest, indicative of congestive heart failure. No animal was alive after age 13 weeks (n=20). Evaluation of the organs showed enlarged liver, reduced kidney size and fluid-filled lungs. The ventricles were smaller and atria were enlarged due to accumulation of thrombi.

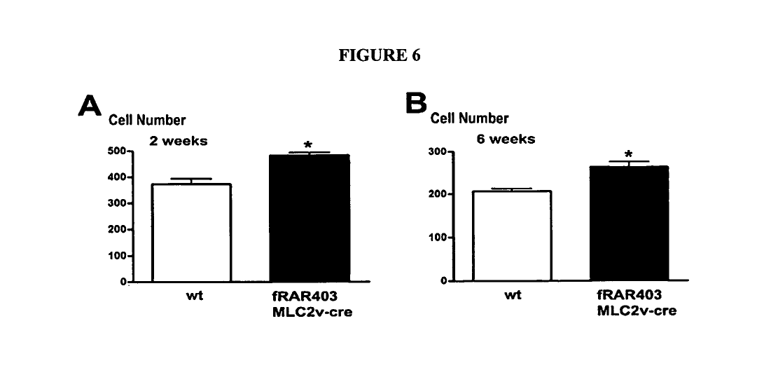

Ablation of retinoid signaling in postnatal myocytes affects myocyte number and sarcomeric structure. fRAR:MLC2vCre mice show normal histology at 2 weeks of age, as displayed by trichrome staining, with no signs of fibrosis although double transgenic mice present a significant increase in the number of myocytes per area unit (FIG. 6A). At six weeks of age, double transgenic present mild fibrosis that was not accompanied by myofibrilar disarray. Of note, the number of myocytes remains significantly higher in the fRAR:MLC2vCre mice compared to wild type controls (FIG. 6B).

Time course for development of heart failure. In order to map the onset of ventricular dysfunction in fRAR403:MLC2vCre mice, we used echocardiography measurements at different postnatal stages. At two weeks RAR403 over-expressers were indistinguishable from controls. At four weeks, hearts of double transgenic mice appeared within normal range and only in comparison to their sex matched littermates did they show a small, but significant decrease in fractional shortening and a small increase in ESD. At four weeks double transgenic were still able to respond equally to increasing amounts of β-adrenergic stimulation just as littermate controls. By ten weeks differences between double-transgenic and wild type mice were obvious, including blunted response to Dobutamine.

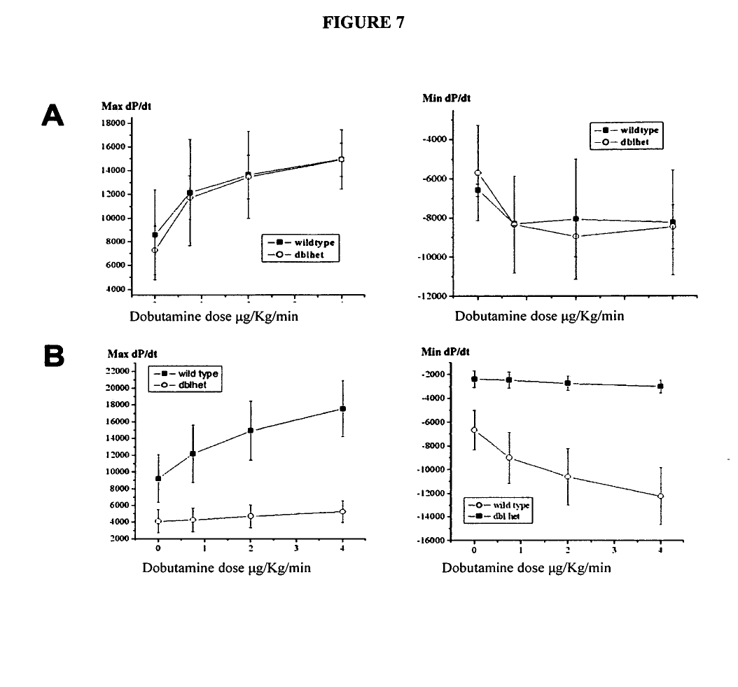

M-mode echocardiography at ten weeks of age show significant decrease in fractional shortening and increase of end systolic (ESD) as well as end diastolic diameters (EDD). There significant decrease in posterior wall and septal wall thickness. Doppler measurements indicated mitral regurgitation in the more severe cases. These mice also displayed a decreased response in contractility and rate of relaxation upon β-adrenergic stimulation (FIG. 7B), common features in human heart failure.

### EXAMPLE 2

### Novel GPCR Expressed in the Heart

GPR22 (20RH) is an orphan type-I GPCR that, at least in adult tissues, is exclusively expressed in the brain and heart, suggesting the 20RH could play a role in cardiac function.

To address its physiological role, we generated conditional mutagenesis of 20RH by means of the generation of a 20RH floxed allele in which exon 2 is flanked by LoxP sites located upstream of a selectable neo FRT. Embryonic stem cell clones positive for homologous recombination were selected by Southern blot and injected in mouse blastocytes to generate mice carrying a floxed allele of 20RH (20RH^{f/+}). Homozygous 20RH floxed mice (20RH^{f/f}) were generated through backcross with parental strain.

In order to establish the physiological role of 20RH, systemic and conditional mutation of 20RH were carried out by means of interbreeding of 20RH^{f/+} with either mice expressing Cre in germ line, protamine Cre (systemic mutant), or expressing Cre in the ventricular myocyte compartment, MLC2v-Cre (ventricular myocyte mutation) (Chen et al., J. Biol. Chem. 273:1252-1256, 1998). Both systemic and ventricular conditional 20RH mutant mice were normal, indistinguishable for their wild-type littermates by all standards measured, including anatomy and lifespan. Furthermore, pressure overload by transaortic constriction failed to show significant differences among 20RH genotypes (Table I). Together, these data demonstrate that mutation in 20RH in the mouse do not result in a basal phenotype or represent disadvantage in pressure overload.

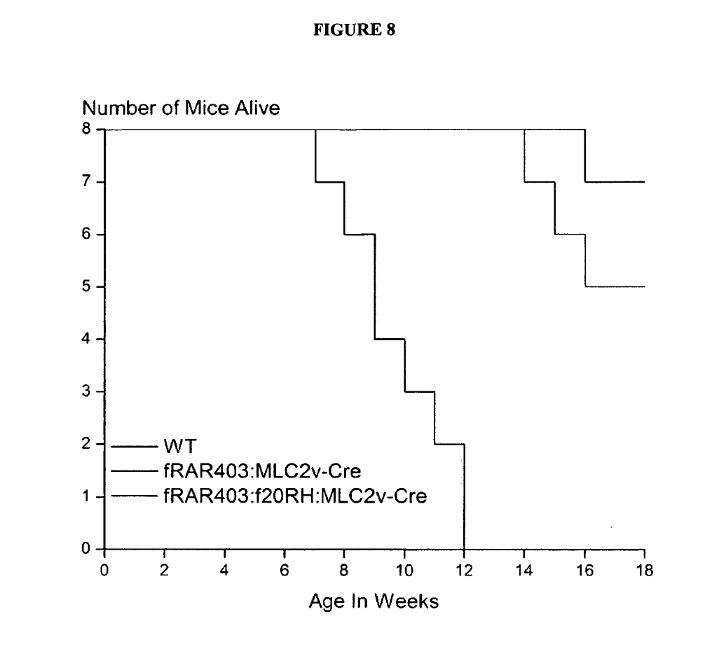

Myocardial-specific mutation of 20RH rescues lethality caused by ablation of retinoid signaling in the myocardium. In searching for genetic modifiers of RAR403 action in myocytes we initiated experiments of genomic complementation using diverse animal models available to us, including 20RH mutant mice. Interbreeding of [fRAR403/+] mice with [20RHf/f: MLC2v-Cre/+] had the following expected progeny: ¼ (RAR403/+; 20RH f/+), ¼ (RAR403/+; 20RHf/+:MLC2v Cre), ¼(+/+; 20RH f/+), and ¼(+/+; 20RH f/+; MLC2vCre). Analysis of the fitness of this set of mutant animals revealed a significant improvement in survival of RAR403 expressing mice in the presence of one single copy of the 20RH coding sequence in the ventricular compartment (FIG. 8).

We have analyzed the survival of eight mice from each genotype, including [fRAR403/+:20RHF/+:MLC2vCre], [fRAR403/+:MLC2vCre], and wild-type controls all of the same black Swiss genetic background.

As previously described, all [fRAR403/+:MLC2vCre] died on or before the 12^{th} week of age. In contrast, all of the [fRAR403/+:20RHF/+:MLC2vCre] were still alive at week 15, of which three of them were found dead between week 15 and 17. Histogical analysis at nine weeks of age demonstrated no signs of dilatation in the “rescued” fRAR403:20HF+/−:MLC2v mice. All of the other [fRAR403/+:20RHF/+:MLC2vCre] mice were still alive after six months of age (24 weeks). In the wild-type controls, one wild-type animal died at 16 weeks of age.

These results demonstrate an improvement of the condition of the transgenic mice upon lowering 20RH expression with significant extension of their lifespan. This strongly suggests that down-regulation of the 20RH mediated signal has a positive impact in cardiac morphology and function.

TABLE 1
Hemodynamic analysis of wt (n = 9) and systemic 20RH mutant mice
(n = 6) at 8 weeks of age before and after aortic constriction (TAC). None of the
parameters measured show significant differences among genotypes.

| | Before TAC | | | After TAC | | |
|---|---|---|---|---|---|---|
| | KO | WT | | KO | WT | |
| | (mean ± SD) | (mean ± SD) | P-value | (mean ± SD) | (mean ± SD) | P-value |
| Number | 12 | 14 | | 6 | 9 | |
| BW (g) | 23.77 ± 2.13 | 23.54 ± 2.76 | 0.167 | 23.70 ± 0.99 | 22.68 ± 1.62 | 0.198 |
| Age (wks) | 12.09 ± 1.21 | 11.51 ± 1.24 | 0.239 | 14.60 ± 1.49 | 13.48 ± 1.12 | 0.135 |
| IVSd (mm) | 0.62 ± 0.03 | 0.61 ± 0.03 | 0.356 | 0.73 ± 0.11 | 0.69 ± 0.07 | 0.370 |
| LVIDd | 3.49 ± 0.30 | 3.53 ± 0.41 | 0.766 | 3.80 ± 0.13 | 3.68 ± 0.60 | 0.622 |
| (mm) | | | | | | |
| LVPWd | 0.62 ± 0.03 | 0.63 ± 0.03 | 0.147 | 0.76 ± 0.15 | 0.70 ± 0.07 | 0.359 |
| (mm) | | | | | | |
| IVSs (mm) | 1.02 ± 0.08 | 1.02 ± 0.07 | 0.980 | 1.13 ± 0.28 | 1.06 ± 0.20 | 0.637 |
| LVIDs | 1.95 ± 0.28 | 1.98 ± 0.36 | 0.779 | 2.53 ± 0.34 | 2.28 ± 0.63 | 0.410 |
| (mm) | | | | | | |
| LVPWs | 1.12 ± 0.09 | 1.13 ± 0.11 | 0.788 | 1.22 ± 0.16 | 1.21 ± 0.17 | 0.967 |
| (mm) | | | | | | |
| HR (bpm) | 461.54 ± 52.63 | 472.62 ± 64.81 | 0.637 | 475.67 ± 59.61 | 457.13 ± 53.02 | 0.550 |
| Ao-ET | 55.38 ± 5.55 | 56.31 ± 5.66 | 0.678 | 58.50 ± 3.39 | 60.63 ± 6.99 | 0.508 |
| (ms) | | | | | | |
| Ao-HR | 437.08 ± 51.47 | 447.54 ± 52.30 | 0.612 | 444.33 ± 62.24 | 437.25 ± 70.08 | 0.848 |
| (bpm) | | | | | | |
| % FS | 44.36 ± 4.22 | 44.13 ± 4.42 | 0.889 | 33.60 ± 8.38 | 38.66 ± 8.76 | 0.298 |
| EDD/PWD | 5.66 ± 0.54 | 5.57 ± 0.63 | 0.712 | 5.18 ± 0.92 | 5.31 ± 1.03 | 0.823 |
| VCF circ/s | 8.10 ± 1.23 | 7.88 ± 0.88 | 0.607 | 5.74 ± 1.39 | 6.41 ± 1.51 | 0.411 |
| LVDd/BW | 0.15 ± 0.02 | 0.15 ± 0.02 | 0.726 | 0.16 ± 0.01 | 0.16 ± 0.03 | 0.879 |
| LVM(d) | 66.94 ± 10.09 | 68.87 ± 13.91 | 0.688 | 98.92 ± 26.43 | 85.16 ± 24.59 | 0.335 |
| (mg) | | | | | | |
| LV/BW | | | | 5.04 ± 0.68 | 5.35 ± 0.58 | 0.316 |
| (mg/g) | | | | | | |
| RV/BW | | | | 0.86 ± 0.11 | 0.87 ± 0.11 | 0.759 |
| (mg/g) | | | | | | |
| H/BW | | | | 6.32 ± 0.86 | 6.62 ± 0.69 | 0.433 |
| (mg/g) | | | | | | |

What is claimed is:

1. A method of identifying a candidate substance that reduces GPR22 activity in a cell comprising: (a) providing a cell comprising a level of GPR22 activity; (b) contacting the cell with the candidate substance; (c) determining whether the candidate substance reduces said level of GPR22 activity.

2. The method of claim 1 wherein the candidate substance is an antagonist of GPR22 G-protein coupled receptor activity.

3. The method of claim 1 wherein the candidate substance reduces levels of GPR22 polypeptide in the cell.

4. The method of claim 3 wherein the candidate substance is selected from the group consisting of an antisense polynucleotide, a ribozyme, and an siRNA molecule.

5. The method of claim 1 wherein the cell is a cardiomyocyte cell, the method comprising determining whether the candidate substance improves survival of the cardiomyocyte cell.

6. The method of claim 5 comprising contacting the cardiomyocyte cell with the candidate substance in vitro.

7. The method of claim 5 comprising determining whether the candidate substance improves survival of the cardiomyocyte cell by measuring apoptosis of the cardiomyocyte cell.

8. The method of claim 1 wherein the cell is a brain cell, the method comprising determining whether the candidate substance improves survival of the brain cell.

9. The method of claim 8 comprising contacting the brain cell with the candidate substance in vitro.

10. The method of claim 8 comprising determining whether the candidate substance improves survival of the brain cell by measuring apoptosis of the brain cell.

11. The method of claim 1 comprising administering the candidate substance to an animal comprising the cell.

12. The method of claim 11 wherein the animal is selected from the group consisting of a mouse, rat, and pig.

13. The method of claim 12 wherein the animal is a mouse or rat.

14. The method of claim 11 wherein the animal is a non-human animal comprising a gene encoding a polypeptide comprising a human GPR22 polypeptide or a fragment thereof having GPCR activity that is expressed in the cell.

15. The method of claim 11 wherein the animal has impaired cardiovascular function resulting from a cardiovascular disease, disorder, or injury.

16. The method of claim 15 wherein the cardiovascular disease, disorder or injury is selected from the group consisting of ischemic heart disease, post-myocardial infarction remodeling, and congestive heart failure.

17. The method of claim 16 wherein the cardiovascular disease, disorder or injury is ischemic heart disease.

18. The method of claim 17 wherein ischemic heart disease is a myocardial infarction.

19. The method of claim 15 wherein the animal is a surgical model of ischemic heart disease or heart failure.

20. The method of claim 11 wherein the animal has impaired neurological function resulting from a disease, disorder, or injury of the brain.

21. A method of preventing or treating a disease, disorder or injury of the heart or brain in an individual in need thereof, the method comprising administering to the individual a therapeutically effective amount of a substance that reduces GPR22 activity.

22. The method of claim 21 wherein the disease, disorder or injury is a disease, disorder or injury of the heart.

23. The method of claim 22 wherein said disease, disorder or injury is characterized by reduced cardiac output or increased venous pressure.

24. The method of claim 22 wherein the disease, disorder or injury is selected from the group consisting of ischemic heart disease, post-myocardial infarction remodeling, and congestive heart failure.

25. The method of claim 22 wherein the disease, disorder or injury is a disease, disorder or injury of the brain.

26. The method of claim 21 wherein the individual is selected from the group consisting of a horse, cow, sheep, pig, cat, dog, rabbit, mouse, rat, non-human primate or human.

27. The method of claim 26 wherein the individual is a human.

28. A composition comprising a polynucleotide selected from the group consisting of an expression vector comprising an GPR22 promoter operably linked to a sequence that, when expressed, reduces GPR22 gene expression in a cell; an siRNA that reduces GPR22 gene expression; an antisense polynucleotide that reduces GPR22 gene expression; and a ribozyme that reduces GPR22 gene expression; wherein the composition is therapeutically effective in treating a disease, disorder or condition of heart or brain of an individual in need thereof.

29. The composition of claim 28 comprising the expression vector wherein the sequence encodes a polynucleotide selected from the group consisting of an siRNA, an antisense polynucleotide, and a ribozyme.

30. The composition of claim 28 comprising a carrier.

31. The composition of claim 28 that is therapeutically effective in treating a disease, disorder or condition of the heart of the individual in need thereof.

32. The composition of claim 28 that is therapeutically effective in treating a disease, disorder or condition of the brain of the individual in need thereof.

33. A method of treating a disease, disorder or injury of the heart or brain in a patient in need of such treatment comprising administering to the patient an effective amount of the composition of claim 28.

34. The method of claim 33 wherein the disease, disorder or injury is a disease, disorder or injury of the heart.

35. The method of claim 27 wherein the disease, disorder or injury is a disease, disorder or injury of the brain.


SEQUENCE LISTING (5 sequences)

SEQ ID NO: 1 - DNA, 5624 bp, Homo sapiens
gtcattctgcaaaagctgcccgaacaggctttcctctgaaaagcagtgccgttcctttttagaaagactgaatctaaatgttgtctctggagacaagaagccttcagtatgttaaattactttcattatgtattttcagatgcttattgattccacagtaggaagagtgagagactgcagcagcctctaagcagcactacatgttccatacattagcagtactgctgaaacaatggcacactacagacacatattttcattaaggtcattttcgaagagatgtttatgaccctcttcccccagtcctctactaacaagtgaacaaaatgaatcaatccaaactggaaatgcttcaactacatcaagaatttatcaaatctttagcagaggtaagatttgttcctattatagtatacagctgcttttgaaccggcatagtggggtaaaaattactttgaaaaatttcagcctaaattttaagagtttgtttaattctacttattgctagacatgtattttaagatctatttcttttaagacatgctacattttaaatgtaattttttaggatgcattgttaaatacaaatattctttgtaaattcattatgaagacagcttttgagtcacttcagatatactaaatattcttaatgtaatcagtacagtttttctccagtgtttcaaaaatgcttctgtttcttaaaagaactaggtttactattttgctgttacttattacttaattttatgttaagtttattatttggcatgttcactgaaaattaatttgcagttacaatttttattaccttttgtatttcaaccacatgaacaattcatcaatggaaatttatagtatcttatgaaatctgttttaatgagttaaaattcaaaccatgtgctaataattaggcaagttacaatatttttagagcaaaaaatgttgttaaatttattacaaagaagaaataacagtatgtttagtgtatgctaattgttcatcatctctgaaaatacaggttagcttctaaaatgagagcagaaattagttttaaaaaatatagcttttctctcacaattgtatttgaaatgtgaactctattgttaaataacataatatatacaatctttattaggaaaaaagacttatttaaagaaacaattttatgcatatagctgtggctctaataataacagtttggttattttgataacaaatactggatagtttttaaacaaaactaaagtaacttgcaattaattacaaattattaaaaaccctatttcttaaccaattttcttcttttcacaagggccaatagcagctaattcagtatttacaactgacaatatgaagaatgcaattgactgagcatctccctagctgtctgaactacgaactgcaagatgttcttgtaacacgactttaagacattaaggagttaaaaccagggaataggtctacattactgatggaatataaaaaatcaactgtatcctaagaagatgctacataaaataaccacaaaaagaaaaacaatataactgtaaaagcctgaaaagaatttttaaaaggggaagtttatactttcatataccagaattgtggaagttactgattctggaagacataatgaaacatgaatttccaaaaagaaaagaaaatactttatcagcacacaaaaggaagatttaggaagtgttttctgcactaaatattcagatattcatatcaattggtatgactaatggatttttctatctgacttttatgaccaattatgtatcctcttctaatgaaaacaaacaaaattaaacagcagatggtttttatcaaaaggacatggcctggatttataatataaagcaagttatgtgatcaagaaatcatttcaaaatagtgagcactgctattaaaaacagatttacaatggtaacaaaaggatgtctaaatatattttagaagctacaaacgttatgtttccttttttgttttcacatattctggaaaataaagaaatattatcatgtactccatcaaagggaaacataattcctatcatctgaggaaattcctcttggccgtgactttttaaagcaaaacaaatacaaatattatgtactgttctttagaaatccatcagccaactaaatctcataatgcatgcagttgaagtattggagagaaaacgaaagaattcctacaagacatgaaataaaacacagctacttcactgttgtcaggtaaaaattcatgtcaaaatctgtcaatgatatcatgtatcaatttgccaaaaactgtcatagtgaaccaaaaggcccataaggcaacagcaaacaggtaggtcagaagatgcatgcaccccaccacactgtttaacatttacgaaagaacagaatcttgctctagagaaaatgtatttttcttaatcatcatttaacttgacatttctgctttatttaattataaatctaactgatgtgacaaagacctgatgtttaatctgtcagttcagaaaatttggcacacttaaaattttccatttttataggatttcaatgttagctaagaccttaacttactcgaataaatatacctctagtaatacttcatactaattcaaaagaaataatgttaccatttgtgtttctgcaatattattcccaaaaaagttcataaataaaactgtattctaaaacttgtcaaatataacattaagtgaaagttaacatgaattttgaaaattattagcttttataatttatgttgaatatgattttgcagttgaaatgctgtatttgaaatgtgaaatatagttggttgtatgagttcatattttaaaatgaatatactgattaactgacattttagcccagaacattagacattattttttacaaattattctaaacccctatatattaaaatatagatttgcatgaattagcaaaagcgtttgtatttttttttaatttttcttcaaaattagacaatgggtgatatatacaaaaggcttcacaggtaatgtgtaactttaaaagcttcctcaaaaaacgggcttcacatactccttttccctaaaatttcatcttttactttaaaaactcagtttaaacaatgtcaactgatatattcctttaaactacttcaaaaatggctatttcttaatgtgatattaaatttcaattttctgtttccacaagatacaagagggtgtggtcacaaaataaatcatttcataggagtgattaatgctcttttaccaagctttgtagctttttattttagcataatgtcccacttcttctaatttttaagtttgccaactgcaaagttcagtgagcattgtaactgtgatatgtaaaatattaatcactggcctatttctgtgggaaataaccccaaatatcaccaagcacattgttgacttctgaatatgaattcaatcaacatggataaagcatcttaaactagtgctctgtaccatttgtcattttaaatgaacacttgtcctgttcatttgaaatctcacaggaataattacacctacactcattatgctagagcatattaaaatagcattcatttggtacctactcacaacatttaaatgaaattttaagacactgggctgaaattaattttgtatgctaggaagttttatcatacaaaaatacactttatctcaaataataagcttgaaatactcaaatgagaaaagccctttagcatattaactttgcactacagaggaacaatttccatagttatttcttcaaaaggaaaacacaattttcttttatatcaaaacaatgcaaacttgatggttcttaattctacattttctattaatagtttacaaacttaaaaattaaactaagtacacaattgaaagattttttttcttacaaagaacacgttatacgtcatttaaattgccaaatatcaaatagtttattctatttcactttctagggaaaaaaaccaactgctccaaaagaatgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttctatagtagaagctgatcccctgcctaataatgctgtaatacacaactcttggatagatcctaaaagaaacaaaaaaattacctttgaagatagtgaaataagagaaaaatgtttagtgcctcaggttgtcacagactagagaaaagtctcagtttcaccaaatccacattcaaatgagttttaaatttaaattgtaaaaactgatattactgccaaatataagaaaaatattttaagtattggttatgttgtaaattttcaatgtgaatgtcaattagataggtcatatatattcaatttcttcattacttaatgtatttgttgcatggcagtttgttaaagtactatcatgtgtatattttgtcaatattatgtccaacagaaaatattcatgtaagtcatattttttaaggaataaatacatagccttaaaacagtgtataactttaaaatgtaa

SEQ ID NO: 2 - DNA, 2976 bp, Homo sapiens
gtcattctgcaaaagctgcccgaacaggctttcctctgaaaagcagtgccgttcctttttagaaagactgaatctaaatgttgtctctggagacaagaagccttcagtatgttaaattactttcattatgtattttcagatgcttattgattccacagtaggaagagtgagagactgcagcagcctctaagcagcactacatgttccatacattagcagtactgctgaaacaatggcacactacagacacatattttcattaaggtcattttcgaagagatgtttatgaccctcttcccccagtcctctactaacaagtgaacaaaatgaatcaatccaaactggaaatgcttcaactacatcaagaatttatcaaatctttagcagagggccaatagcagctaattcagtatttacaactgacaatatgaagaatgcaattgactgagcatctccctagctgtctgaactacgaactgcaagatgttcttgtaacacgactttaagacattaaggagttaaaaccagggaataggtctacattactgatggaatataaaaaatcaactgtatcctaagaagatgctacataaaataaccacaaaaagaaaaacaatataactgtaaaagcctgaaaagaatttttaaaaggggaagtttatactttcatataccagaattgtggaagttactgattctggaagacataatgaaacatgaatttccaaaaagaaaagaaaatactttatcagcacacaaaaggaagatttaggaagtgttttctgcactaaatattcagatattcatatcaattggtatgactaatggatttttctatctgacttttatgaccaattatgtatcctcttctaatgaaaacaaacaaaattaaacagcagatggtttttatcaaaaggacatggcctggatttataatataaagcaagttatgtgatcaagaaatcatttcaaaatagtgagcactgctattaaaaacagatttacaatggtaacaaaaggatgtctaaatatattttagaagctacaaacgttatgtttccttttttgttttcacatattctggaaaataaagaaatattatcatgtactccatcaaagggaaacataattcctatcatctgaggaaattcctcttggccgtgactttttaaagcaaaacaaatacaaatattatgtactgttctttagaaatccatcagccaactaaatctcataatgcatgcagttgaagtattggagagaaaacgaaagaattcctacaagacatgaaataaaacacagctacttcactgttgtcagggaaaaaaaccaactgctccaaaagaatgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttctatagtagaagctgatcccctgcctaataatgctgtaatacacaactcttggatagatcctaaaagaaacaaaaaaattacctttgaagatagtgaaataagagaaaaatgtttagtgcctcaggttgtcacagactagagaaaagtctcagtttcaccaaatccacattcaaatgagttttaaatttaaattgtaaaaactgatattactgccaaatataagaaaaatattttaagtattggttatgttgtaaattttcaatgtgaatgtcaattagataggtcatatatattcaatttcttcattacttaatgtatttgttgcatggcagtttgttaaagtactatcatgtgtatattttgtcaatattatgtccaacagaaaatattcatgtaagtcatattttttaaggaataaatacatagccttaaaacagtgtataactttaaaatgtaaaaaaaaaaaa

SEQ ID NO: 3 - protein, 433 aa, Homo sapiens
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMISIWIFSFFSFLIPFIEVNFFSLQSGNTWENKTLLCVSTNEYYTELGMYYHLLVQIPIFFFTVVVMLITYTKILQALNIRIGTRFSTGQKKKARKKKTISLTTQHEATDMSQSSGGRNVVFGVRTSVSVIIALRRAVKRHRERRERQKRVFRMSLLIISTFLLCWTPISVLNTTILCLGPSDLLVKLRLCFLVMAYGTTIFHPLLYAFTRQKFQKVLKSKMKKRVVSIVEADPLPNNAVIHNSWIDPKRNKKITFEDSEIREKCLVPQVVTD

SEQ ID NO: 4 - DNA, 30 bp, Artificial Sequence
  Description of Artificial Sequence Synthetic primer
tcccccgggaaaaaaaccaactgctgcaaa

SEQ ID NO: 5 - DNA, 31 bp, Artificial Sequence
  Description of Artificial Sequence Synthetic primer
taggatccatttgaatgtggatttggtgaaa